(19) United States
(12) Patent Application Publication
(10) Pub. No.: US 2026/0339914 A1
(43) Pub. Date: Oct. 29, 2026

(54) NODE MODIFICATIONS FOR GENEALOGICAL TREES BASED ON GENETIC MATCHES

(71) Applicant: Ancestry.com Operations Inc., Lehi, UT (US)

(72) Inventors: Kenric Lloyd Russell, American Fork, UT (US); Brett Frederick Jorgensen, Draper, UT (US); Bryce Damon Ririe, Highland, UT (US); Manun Chopra, San Francisco, CA (US); Brooke Ann Alius, Lehi, UT (US); Kelly McCloy Becker, Salt Lake City, UT (US); Gorkem Eskici, San Francisco, CA (US); Barry Watts, Highland, UT (US)

(21) Appl. No.: 19/190,444

(22) Filed: Apr. 25, 2025

(51) Int. Cl. G06F 16/22

(52) U.S. Cl. CPC G06F 16/2246

(57) ABSTRACT

The present disclosure relates to systems, non-transitory computer-readable media, and methods for generating or modifying genealogical trees based on genetic match data. In particular, in one or more embodiments, the disclosed systems determine genetic-similarity predictions between user accounts of a genealogical-data system and generate a genetic match from the genetic-similarity predictions. In one or more embodiments, the disclosed systems determine a tree connection between individuals linked by the genetic match and further provide a notification to modify one or more genealogical trees based on determining the tree connection associated with the genetic match.

### BACKGROUND

Advancements in computing devices and networking technology have given rise to a variety of innovations in cloud-based genealogical data storage, sharing, and generation. For example, online historical-content systems can provide access to digital genealogical records across devices all over the world. Online historical-content systems can also analyze genealogical data for specific user accounts to generate links or relationships between accounts and genealogical records. In addition, historical-content systems often integrate with genetic-analysis systems that analyze genetic material and provide insights into global origins and heritage to user accounts associated with genetic material. Existing genetic-analysis systems can analyze genetic material to determine genetic matches that indicate the biological relationships of individuals associated with user accounts. Despite these advances, however, existing systems continue to exhibit a number of disadvantages, particularly in terms of flexibility and the number interactions required to update genealogical data in genealogical trees.

As just suggested, certain existing systems are inflexible. More particularly, some existing systems generate genealogical trees with nodes that represent individuals and/or user accounts and edges representing relationships between the nodes. Other existing systems can identify genetic similarities between a user account and other individuals using a sample of genetic material. However, existing systems often isolate genetic-matching analysis apart from genealogical-tree management, using separate platforms and/or databases for each process. Indeed, existing systems provide information regarding genetic matches and information regarding genealogical trees in separate siloed locations (e.g., separate applications or interfaces).

In part due to their inflexibility, some existing systems also require excessive numbers of user interface interactions to manage genealogical trees (e.g., to add and/or modify nodes for genetic matches). Specifically, because many systems isolate genetic data apart from genealogical tree data, users must perform excess user interface interactions navigating between multiple interfaces and/or applications, analyzing genetic matches and adding select genetic matches to a genealogical tree. In some cases, to add a genetic match to a genealogical tree, users must often sift through match data of a genetic-analysis system to identify a match, search for a user account corresponding to a genetic match in a user account database and further perform a series of user interface interactions to add a node for the genetic match to a genealogical tree. This problem is exacerbated when genealogical trees for matched individuals do not share nodes, resulting in further interactions to find common ancestors to link the trees with a shared node.

In addition, existing systems are also inaccurate. In particular, some existing systems rely solely on record data from historical documents, such as birth certificates, census data, and marriage licenses, to generate genealogical trees. Genealogical trees in such systems often include missing, incorrect, or incomplete branches and/or nodes due to missing, incomplete, or inaccurate historical records, such as due to war or natural disaster, or simply due to lack of recordkeeping. Further, errors in existing systems are perpetuated through multiple genealogical trees as existing systems often utilize a shared genealogical database, where genealogical trees share information by adding information (or a node) from one genealogical tree to another genealogical tree. Thus, even a single error can lead to widespread inaccuracies in existing systems. Further, where facts regarding tree persons rely on user-generated data, such as user-generated dates, places, and names, human errors due to misapprehensions by descendants of tree persons can exacerbate the challenges associated with linking tree persons.

In addition, existing systems are not intuitively accessible for users, particularly new users. For example, unless they have extensive exposure to genealogical research, users are unlikely to have an intuitive appreciation for relationship labels such as “second cousin once removed” or “third cousin” when presented in isolation. Such users are liable to lose interest in and stop engaging with a genealogical research service, thereby arresting any further meaningful discoveries that they might have otherwise made, as they are presented with and urged to proceed through a morass of DNA matches with such non-intuitive relationship labels.

### SUMMARY

This disclosure describes one or more embodiments of systems, methods, and non-transitory computer-readable storage media that provide benefits and/or solve one or more of the foregoing and other problems in the art. For instance, the disclosed systems determine a genetic match between individuals or user accounts as a basis for updating or modifying genealogical trees of the user accounts. For example, the disclosed systems determine a genetic match and directly integrate the genetic match into a node (or a suggestion for adding or updating a node) of a genealogical tree for a user account of an individual involved in the match. In one or more embodiments, the disclosed systems determine and consider additional factors, including tree connections between individuals that share a genetic match, as part of modifying (or providing notifications to modify) genealogical trees.

### BRIEF DESCRIPTION OF THE DRAWINGS

The detailed description provides one or more embodiments with additional specificity and detail through the use of the accompanying drawings, as briefly described below.

FIG. 1 illustrates a diagram of an environment in which a genetic-match-suggestion system can operate in accordance with one or more embodiments.

FIG. 2 illustrates an example diagram of an overview of a genetic-match-suggestion system determining a tree connection between a first user account and a second user account and providing a node suggestion for the first user account for a genealogical tree in accordance with one or more embodiments.

FIG. 3 illustrates a schematic diagram of a genetic-match-suggestion system identifying a tree connection for a suggested node and adding a node to a genealogical tree through the tree connection in accordance with one or more embodiments.

FIGS. 4A-4B illustrate a schematic diagram of a genetic-match-suggestion system providing node suggestions to add intermediate nodes to a genealogical tree to establish a tree connection to a shared node in accordance with one or more embodiments.

FIGS. 5A-5G illustrate example graphical user interfaces of the genetic-match-suggestion system providing and displaying node suggestions to add to a genealogical tree in accordance with one or more embodiments.

FIGS. 6A-6C illustrate example graphical user interfaces of the genetic-match-suggestion system updating existing nodes of a genealogical tree with genetic data in accordance with one or more embodiments.

FIGS. 7A-7B illustrates example graphical user interfaces for providing a preview of a location of a node suggestion within a genealogical tree in accordance with one or more embodiments.

FIG. 8 illustrates a block diagram of an example computing device for performing genetic and genealogical analysis in accordance with one or more embodiments.

FIG. 9 illustrates a tree database and a cluster database of the genealogical agentic system in accordance with one or more embodiments.

FIG. 10 illustrates a flowchart of a series of acts for generating genealogical matches and providing node suggestions for genealogical trees in accordance with one or more embodiments.

FIG. 11 illustrates a block diagram of an example computing device for implementing one or more embodiments of the present disclosure.

FIG. 12 illustrates an example environment of a networking system with the genetic-match-suggestion system in accordance with one or more embodiments.

FIG. 13 illustrates an example graphical user interface for generating a relationship path between a user and a genetic match in accordance with one or more embodiments.

FIG. 14 illustrates another example graphical user interface for generating a relationship path between a user and a genetic match in accordance with one or more embodiments.

FIG. 15 illustrates another graphical user interface for generating a relationship path between a user and a genetic match in accordance with one or more embodiments.

FIG. 16 illustrates another graphical user interface for generating a relationship path between a user and a genetic match in accordance with one or more embodiments.

FIG. 17 illustrates another graphical user interface for generating a relationship path between a user and a genetic match in accordance with one or more embodiments.

### DETAILED DESCRIPTION

This disclosure describes one or more embodiments of a genetic-match-suggestion system that determines a genetic match between a first user account and a second user account, and which provides a suggestion to the first user account to add (or which automatically adds) the second user account to a genealogical tree. Specifically, the genetic-match-suggestion system generates genetic-similarity predictions indicating similarities between a genetic profile of the first user account and genetic profiles of additional user accounts. From the genetic-similarity predictions, the genetic-match-suggestion system determines the genetic match, indicating that an individual represented by the second user account matches genetically an individual represented by the first user account. Based on the genetic match, the genetic-match-suggestion system establishes there is a tree connection between the first user account and the second user account and provides a node suggestion for the first user account to add a node corresponding to the second account to an identified pertinent location in an associated genealogical tree. Based on receiving a user interaction with the node suggestion, the genetic-match-suggestion system adds (or modifies) a node corresponding to the second user to account to the pertinent location in the associated genealogical tree.

As just mentioned, as the basis for generating a node suggestion, the genetic-match-suggestion system determines that there is a tree connection between a first account and a second user account. In particular, the genetic-match-suggestion system compares a genealogical tree for or associated with the first user account to a genealogical tree for or associated with the second user account to identify a shared node (e.g., an ancestor, such as a most-recent common ancestor, in both genealogical trees) and identifies a tree connection between the shared node and the second user account. In some cases, a tree connection between a shared node and the second user account is a single-generation connection, where the second user account is directly connected (with zero intervening nodes or zero steps removed) to the shared node (e.g., in a parent-child or sibling relationship). In other cases, the second user account and the shared node have an intermediate connection where there is a node (or multiple nodes) between the shared node and the second user account (e.g., the second user account is a grandchild, great-grandchild, or cousin of the shared node).

In one or more embodiments, the genetic-match-suggestion system provides a node suggestion with an indication of a tree connection. For example, the genetic-match-suggestion system provides a visualization depicting the tree connection between the second user account and the first user account, such as if there is a single-generation connection to a shared node or if there is an intermediate connection to the shared node through one or more intermediate nodes. Moreover, in some embodiments, the genetic-match-suggestion system provides node suggestions and/or visualizations of tree connections based on receiving a user interaction to provide user accounts with a genetic connection to the first user account. In some cases, the genetic-match-suggestion system provides the node suggestion based on receiving a user selection of an option to provide genetic matches with a specific node (e.g., the shared node) of the genealogical tree of the first user account.

In one or more embodiments, the genetic-match-suggestion system adds a node to a genealogical tree based on a genetic match (or based on receiving a user interaction with the node suggestion for a genetic match). In addition, in some embodiments, the genetic-match-suggestion system provides a genealogical record for a shared node and requests confirmation that information in the genealogical record corresponds to the shared node. If the first user account provides an indication that the genealogical record corresponds to the shared node, the genetic-match-suggestion system adds the node corresponding to the second user account to the genealogical tree. In some cases, the genetic-match-suggestion system provides a node suggestion for the first user account to add an intermediate node (e.g., a connecting node) to the genealogical tree. In addition, the genetic-match-suggestion system provides a node suggestion for the first user account to add a node corresponding to the second user account based on the first user account adding the intermediate node to the genealogical tree.

The genetic-match-suggestion system provides a variety of technical advantages relative to conventional systems. For example, the genetic-match-suggestion system provides unique functionality for a new tree-building experience not found in prior systems. To elaborate, the genetic-match-suggestion system provides tools and interface elements for users to continue building, expanding, and adding information to genealogical trees. In contrast with prior systems that limit genealogical-tree building to individual knowledge and/or record-based suggestions, the genetic-match-suggestion system can further integrate genetic data into the tree-building experience. Consequently, the genetic-match-suggestion system provides an entirely new (and very large) set of data for informing and integrating into genealogical trees.

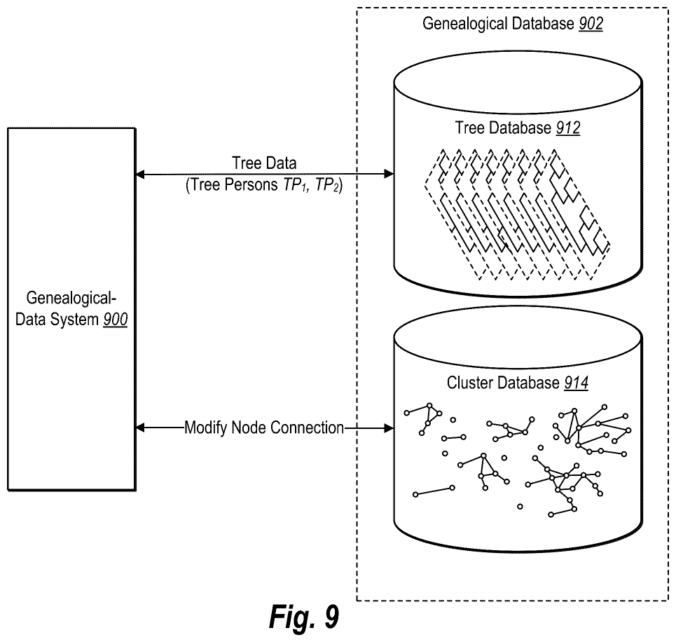

Not only does the genetic-match-suggestion system thus generate more robust genealogical trees with additional genetic data to inform, corroborate, and/or correct nodes and edges (e.g., to avoid incorrectly merging nodes of distinct individuals), but the genetic-match-suggestion system further expands genealogical trees to include additional relationships not found in existing records (and which therefore cannot be added in prior systems), both laterally in existing generations and/or vertically for new generations. For example, the genetic-match-suggestion system can leverage genetic data to identify a long-lost relative whose relationship does not occur in stored genealogical records. The genetic-match-suggestion system can thus link a user account with the long-lost relative and can further analyze genealogical tree data for the long-lost relative (in cases where the long-lost relative also has a user account and has built a genealogical tree) to supplement tree data in the genealogical tree of the user account. As another example, the genetic-match-suggestion system can utilize the genetic similarity of the long-lost relative to identify genealogical records (maintained by the long-lost relative) that are relevant to the user account. Indeed, the genetic-match-suggestion system can modify a cluster database—e.g., a database storing clusters of genealogical records (e.g., where each cluster corresponds to an individual) that inform a universal genealogical tree maintained by the genealogical-data system—to link the user account with a previously unaffiliated cluster of records corresponding to the long-lost relative. Additional details and examples of a cluster database are provided below with respect to FIG. 9.

As a result of providing a new tree-building experience, by providing node suggestions within a genealogical tree to add nodes for genetic matches, the genetic-match-suggestion system improves flexibility relative to conventional systems. Specifically, unlike prior systems that isolate genetic analysis and genealogical record analysis (e.g., genealogical trees), the genetic-match-suggestion system integrates genetic matches directly with genealogical trees by combining genetic-match-based node suggestions for modifying nodes and/or edges within record-based genealogical trees. As part of integrating genetic-match analysis with genealogical trees, the genetic-match-suggestion system also determines shared nodes and tree connections among individuals indicated in a genetic match. Further, the genetic-match-suggestion system determines additional and/or alternative tree connections to establish a tree connection and provides indications for intermediate nodes forming the tree connection. Indeed, the genetic-match-suggestion system can flexibly integrate genetic-analysis systems and a genealogical record system to determine locations and connections within a genealogical tree.

In addition, the genetic-match-suggestion system reduces the number of user interface interactions required to add nodes to a genealogical tree. More specifically, unlike online historical-content systems that require a user to perform excess user interface interactions identifying genetic matches, searching for user accounts corresponding to the genetic match, adding a node for the user account, the genetic-match-suggestion system provides the node directly within the genealogical interface with an option to add the node (e.g., in a single interaction). For example, the genetic-match-suggestion system provides an option that, when selected, allows the genetic-match-suggestion system to add the node to the genealogical tree. Moreover, rather than requiring a user to perform excessive user interface interactions searching for connections required to add a node for a genetic match (across multiple interfaces and/or applications), the genetic-match-suggestion system consolidates genetic-match analysis and genealogical-tree information into a single interface for providing node suggestions. As part of the consolidation of interface functionality, the genetic-match-suggestion system further provides information and/or node suggestions for intermediate nodes connecting individuals of a genetic match, where the intermediate nodes can also be added by selecting options, greatly reducing the number of interactions compared to prior systems that require searching separate databases (e.g., a genetic match database and a record database) and individually adding each intervening node. Further, after adding the intermediate nodes, the genetic-match-suggestion system automatically adds an option to a node suggestion to add a node for the genetic match easily with a single user interface interaction.

Further, by incorporating genetic matches into genealogical trees, the genetic-match-suggestion system improves accuracy relative to existing systems. Specifically, by utilizing genetic matches in conjunction with tree connection in genealogical trees, the genetic-match-suggestion system can leverage genetic matches and display node suggestions that reconcile or remedy incomplete, missing, or inaccurate information in trees of existing systems. Indeed, the genetic match suggestion can generate or modify nodes based on shared genetic data and based on determining tree connections relating to the shared genetic data. Thus, by combining genetic match data with genealogical tree data, the genetic-match-suggestion system can more accurately and reliably add or modify nodes of genealogical trees than prior systems.

As illustrated by the foregoing discussion, the present disclosure utilizes a variety of terms to describe the features and advantages of the genetic-match-suggestion system. Additional details regarding the meaning of such terms are now provided.

For example, as used herein, the term “genealogical tree” refers to a structured representation of relationships between individuals across generations within a family, e.g. a pedigree represented in an online genealogical research service. In particular, the term “genealogical tree” includes a structured hierarchical format for representing familial relationships to provide a clear and organized understanding of family relationships and lineage. A genealogical tree can be part of a genealogical-data system that leverages database systems and/or algorithmic tools to facilitate data storage, retrieval, or visualization of genealogical trees. Information and/or individuals can be added to genealogical trees using metadata, genetic data, historical records, content items, or genealogical records. In some cases, a genealogical tree is specific to a user account, with relationships depicting ancestors and/or descendants of an individual associated with the user account. Moreover, a genealogical tree can be connected to a broader database of a genealogical-data system, which can identify user accounts that share individuals in a genealogical tree for collaborative editing and sharing of information, data, content items, and other genealogical records. For example, the genealogical-data system may comprise or be configured to cooperate with a cluster database and/or a genealogical-tree database, where nodes of different users' genealogical trees that represent a same individual are resolved together, with media, data, and genealogical trees related to the clustered-together nodes becoming accessible therethrough to other users of the genealogical research system.

In addition, as used herein, the term “node suggestion” refers to data or information that is associated with a genealogical tree of a user account and is provided to the user account as a recommendation to add the associated data or information, such as a node, to the genealogical tree. Specifically, a “node suggestion” refers to recommended information, data, user accounts, or nodes that are associated with a genealogical tree of a user account through a shared genealogical link, such as ancestors, genetic or biological data, demographic information, historical events, historical records, family structures, historical context, digital media or documents, narratives, or timelines. For example, a node suggestion can include a recommendation to add a node for a user account or individual to a genealogical tree. As another example, a node suggestion can include a recommendation to add or update information to an existing node of a genealogical tree.

Moreover, as used herein, the term “shared node” refers to a node that is present in genealogical trees of multiple user accounts. In particular, a “shared node” refers to a node that represents a person in two (or more) genealogical trees and serves as a point of intersection between genealogical trees of two (or more) user accounts (or individuals associated with the user accounts). For example, a shared node is a common data entity with identical or reconcilable attributes, such as name, birth and/or death dates, and relationships that represent a point of intersection of genealogical trees between multiple user accounts. As described above regarding the definition of “genealogical tree,” shared nodes may be resolved together into clusters of a cluster database.

Also, as used herein, the term “intermediate node” refers to a node of a genealogical tree that is a relational intermediary between nodes. Specifically, “intermediate node” refers to an intervening node that establishes a connection between nodes due to familial relationships of the intermediate node with other nodes. For example, an intermediate node imay be a descendant of a node and an ancestor of another node. To illustrate, for a node of a user account and a node of an ancestor for the user account, an intermediate node can correspond to a parent to the user account and a child of the ancestor. As between siblings, an intermediate node can correspond to a shared parent of the two siblings.

In addition, as used herein, the term “genetic profile” refers to a digital representation of the unique genetic markers and/or other genomic features derived through genetic analysis of an individual. In particular, a “genetic profile” refers to a dataset comprising unique genetic markers and/or other genomic features that are derived from genetic analysis (e.g., DNA analysis through microarray genotyping, identical-by-descent analysis, and/or downstream manipulations such as haplotype phasing) of a genetic sample from the individual and provide information about lineage, biological traits, and potential health risks. For example, a genetic profile can include unique genetic variations or polymorphisms, specific genetic sequences of DNA, such as single nucleotide polymorphisms (SNPs), short tandem repeats (STRs), haplogroups, or other genetic markers. In embodiments, a genetic profile for an individual corresponding to a user account comprises an unphased diploid dataset, in embodiments the genetic profile comprises a dataset comprising phased haplotypes. Moreover, in some cases, genetic profiles can also include digital representations of genetic markers and/or other genomic features, such as genetic variations identified during microarray genotyping and respective manipulations (e.g., haplotype phasing) of the genetic variations stored as digital representations.

Further, as used herein, the term “genetic match” refers to the presence of shared genetic material between two individuals, as identified through DNA analysis. Specifically, a “genetic match” refers to shared DNA between individuals that indicates the individuals have a biological relationship. For example, a genetic match is determined by comparing segments of DNA, such as single nucleotide polymorphisms (SNPs) or identical-by-descent (IBD) regions, across the genetic material. The degree of matching is measured in units such as centimorgans (cM), which quantify the amount of shared DNA and provide an estimate of the closeness of the biological relationship. High levels of shared DNA typically indicate a closer relationship, while lower levels suggest a more distant one.

In addition, as used herein, the term “genetic-similarity prediction” refers to a computation or statistical estimate that quantifies the degree to which two individuals share genetic information. Specifically, a “genetic-similarity prediction” refers to a score, percentage, or probabilistic value that indicates a genetic similarity between genetic profiles of user accounts. For example, a genetic-similarity prediction indicates a likelihood that individuals associated with the user accounts are biologically related based on an amount of genetic material shared between the user accounts (e.g., shared IBD segments measured in cM).

Also, as used herein, the term “tree connection” refers to links or relationships that represent the associations between entities or nodes of a genealogical tree. In particular, a “tree connection” refers to the edges within a hierarchical structure of a genealogical tree that represent defined relationships between nodes of a genealogical tree. For instance, a tree connection indicates a relational data point that defines the structures and topology of a genealogical tree. In some cases, a tree connection indicates that there is a familial or biological connection between nodes, including instances where there are several generations between the nodes.

Similarly, as used herein, the term “single-generation connection” refers to a direct connection between nodes. Specifically, a “single-generation connection” is a connection between nodes where there is not a generation between nodes. For example, a single-generation connection could be a parent-child connection or a sibling connection.

In addition, as used herein, the term “genealogical record” refers to a digital object or a digital file that includes information (e.g., genealogical information) interpretable by a computing device (e.g., a client device) to present information to a user. A genealogical record can include a file such as a digital text file, a digital image file, a digital audio file, a webpage, a website, a digital video file, a web file, a link, a digital document file, or some other type of file or digital object. A genealogical record can have a particular file type or file format, which may differ for different types of digital content items (e.g., digital documents. digital images, digital videos, or digital audio files). In some cases, a genealogical record can refer to a content item that includes or depicts historical or genealogical information, such as a digitized birth, marriage, or death record, a digitized newspaper article, a digitized photograph of a relative, a digitized census record, a digitized obituary, a digitized court document, a digitized family history book, or a digitized family tree. In some embodiments, a genealogical record includes a content item selected or identified to surface to a client device, such as a record hint (e.g., a stored genealogical record), a digital story (e.g., a stored genealogical content item or collection of genealogical content items arranged for a particular person, topic, or entity of a genealogical-data system), a digital image (e.g., a digitized photograph), a node suggestion (e.g., a node to add to a genealogical tree), or a genetic match (e.g., a record indicating a DNA match of a user account to a relative whose information is stored in a genealogical-data system).

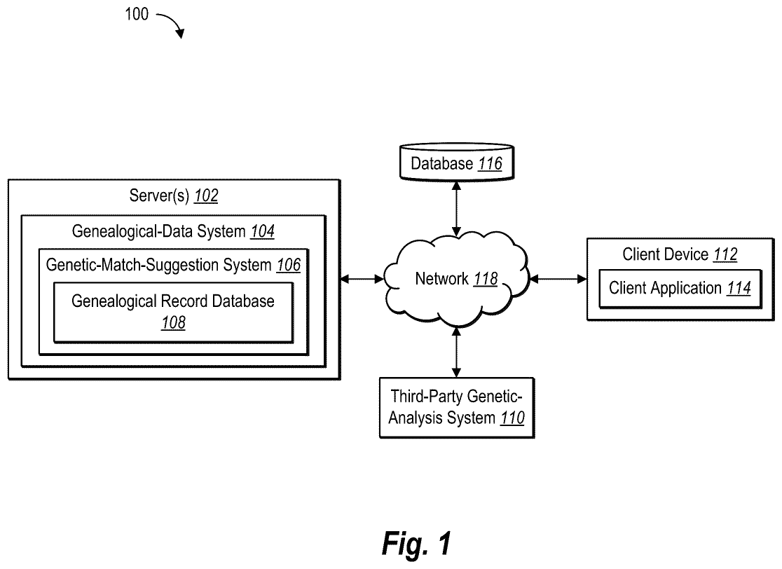

Additional details regarding the genetic-match-suggestion system will now be provided with reference to the figures. For example, FIG. 1 illustrates a diagram of an environment 100 in which a genetic-match-suggestion system 106 can operate in accordance with one or more embodiments. An overview of the genetic-match-suggestion system 106 is provided in relation to FIG. 1. Thereafter, a more detailed description of the components and processes of the genetic-match-suggestion system 106 is provided in relation to the subsequent figures.

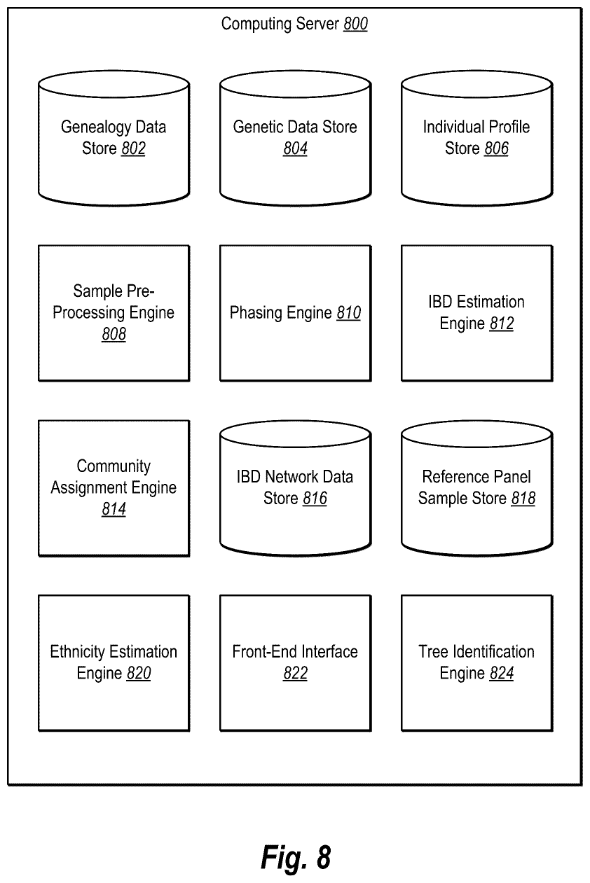

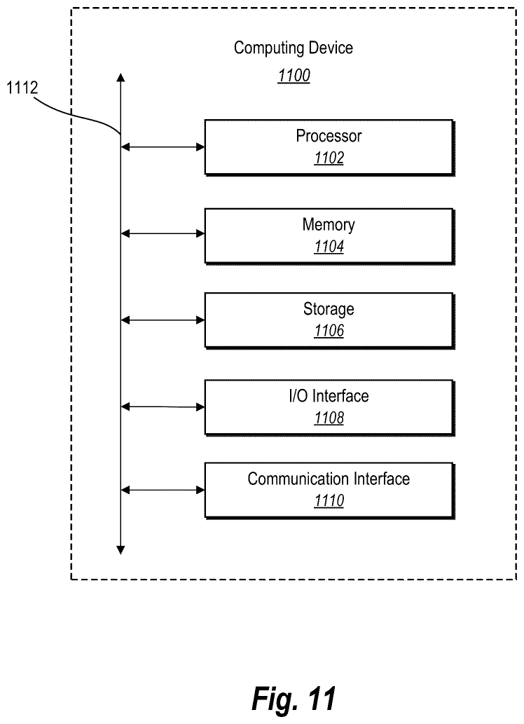

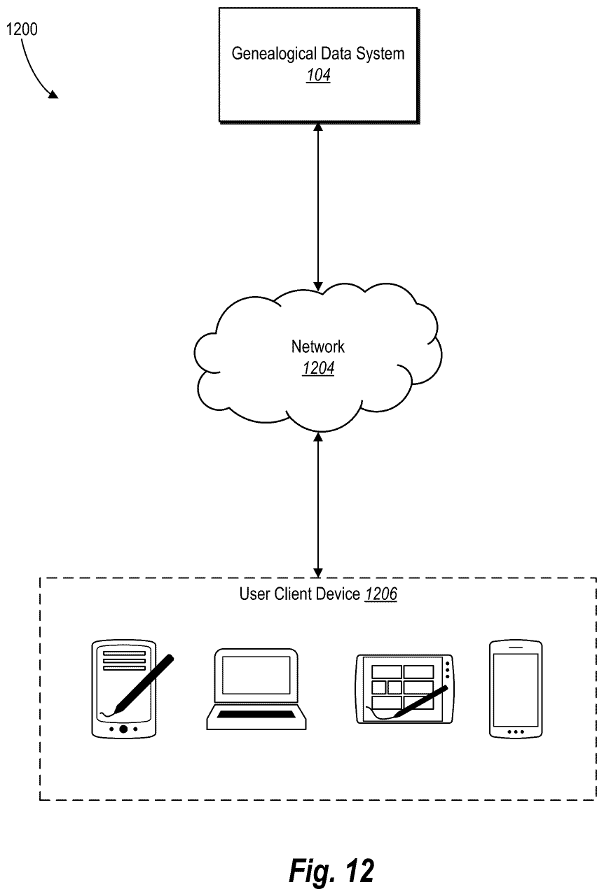

As shown, the environment 100 includes server(s) 102, database 116, third-party genetic-analysis system 110, and client device 112. Each of the components of environment 100 can communicate via network 118, and network 118 may be any suitable network over which computing devices can communicate. Example networks are discussed in more detail in relation to FIGS. 11-12. As mentioned above, environment 100 includes client device 112. The client device 112 can be one of a variety of computing devices, including a smartphone, a tablet, a smart television, a desktop computer, a laptop computer, a virtual reality device, an augmented reality device, or another computing device as described in relation to FIG. 8 and/or FIGS. 11-12. The client device 112 can communicate with the server(s) 102 via network 118. For example, client device 112 can receive user input from a user interacting with client device 112 (e.g., via the client application 114) to, for instance, receive user interactions requesting genetic matches, requesting nodes added to a genealogical tree, or confirming genealogical records. In addition, the genetic-match-suggestion system 106 or the server(s) 102 can receive information relating to various interactions with genealogical content items and/or user interface elements based on the input received by the client device 112.

In one or more embodiments, client device 112 includes client devices associated with various different user accounts. For example, a first client device can be associated with a first user account, and a second client device can be associated with a second user account. In some cases, the first client device displays a node suggestion corresponding to the second user account.

As shown, client device 112 can include a client application 114. In particular, the client application 114 may be a web application, a native application installed on the client device 112 (e.g., a mobile application, a desktop application, etc.), or a cloud-based application where all or part of the functionality is performed by the server(s) 102. Based on instructions from the client application 114, the client device 112 can present or display information, including a user interface for interacting with genealogical trees, including nodes of genealogical trees and/or genealogical records. Using the client application 114, the client device 112 can perform (or request to perform) various operations, such as displaying suggested nodes for a genealogical tree.

As illustrated in FIG. 1, the environment 100 also includes the server(s) 102 (e.g., hardware server devices). The server(s) 102 may generate, track, store, process, receive, and transmit electronic data, such as interface elements and/or interactions between user accounts or client devices. For example, the server(s) 102 may receive an indication from the client device 112 of a user interaction requesting genetic matches, requests to add a suggested node to a genealogical tree, or a confirming a genealogical record corresponds to a node or shared node. In addition, server(s) 102 can transmit data to the client device 112 in the form of a genealogical tree, a suggested node, or a genealogical record. Indeed, the server(s) 102 can communicate with the client device 112 to send and/or receive data via network 118. In some implementations, the server(s) 102 comprise(s) a distributed server where the server(s) 102 include(s) a number of server devices distributed across the network 118 and located in different physical locations. The server(s) 102 can comprise one or more content servers, application servers, container orchestration servers, communication servers, web-hosting servers, machine learning servers, and other types of servers.

As shown in FIG. 1, the server(s) 102 can also include the genetic-match-suggestion system 106 as part of the genealogical-data system 104. The genealogical-data system 104 can communicate with the client device 112 to perform various functions associated with the client application 114, such as managing and adding nodes to genealogical trees. Indeed, genealogical-data system 104 can include a network-based smart cloud storage system to manage, store, and maintain content items and related data across numerous user accounts. For instance, the genetic-match-suggestion system 106 can utilize genealogical data across various content items and user accounts to generate and maintain a universal genealogical tree that reflects the relatedness or consanguinity between nodes corresponding to all user accounts and other individuals indicated by stored genealogical content items. In some embodiments, the genetic-match-suggestion system 106 and/or the genealogical-data system 104 utilize the database 116 to store and access information and/or data corresponding to genealogical trees or genealogical records.

Further, as also shown, the genetic-match-suggestion system 106 can also house the genealogical-record database 108. The genealogical-record database 108 can store or house genealogical information for user accounts, including data and information corresponding to nodes of a genealogical tree and genealogical records for the user account and other nodes of the genealogical tree.

Moreover, as also shown in FIG. 1, the environment 100 can also include a third-party genetic-analysis system 110. The third-party genetic-analysis system 110 receives genetic samples (e.g., saliva samples) and extracts genetic material from the sample to generate a genetic profile for a user account of the genealogical-data system 104. Server(s) 102 (or genealogical-data system 104) can communicate with the third-party genetic-analysis system 110 to receive genetic profiles to associate with user accounts of the genealogical-data system 104 (or the genetic-match-suggestion system 106). In some cases, the third-party genetic-analysis system 110 provides a genetic profile that includes raw genotyping reads (or digital indications of raw genotyping reads) corresponding to user profiles of the genealogical-data system 104. In addition, based on the genetic profile from the third-party genetic-analysis system 110 (or based on analyzing the genetic profile), the genetic-match-suggestion system 106 communicates with a client device 112 to provide indications of a genetic profile (e.g., heritage or global origins), provide information for user accounts of a genetic match, facilitate communication between genetic matches, and other functions.

Although FIG. 1 depicts the genetic-match-suggestion system 106 located on the server(s) 102, in some implementations, the genetic-match-suggestion system 106 may be implemented by (e.g., located entirely or in part on) one or more other components of the environment 100. For example, the genetic-match-suggestion system 106 may be implemented as part of client device 112 and/or a third-party system. As another example, the client device 112 and/or a third-party system can download all or part of the genetic-match-suggestion system 106 for implementation independent of, or together with, the server(s) 102.

In some implementations, though not illustrated in FIG. 1, environment 100 may have a different arrangement of components and/or may have a different number or set of components altogether. For example, client device 112 may communicate directly with the genetic-match-suggestion system, bypassing network 118. The environment 100 may also include one or more third-party systems, each corresponding to a different data source. In addition, the environment 100 can include the database 116 located external to the server(s) 102 (e.g., in communication via the network 118) or located on the server(s) 102 and/or on the client device 112.

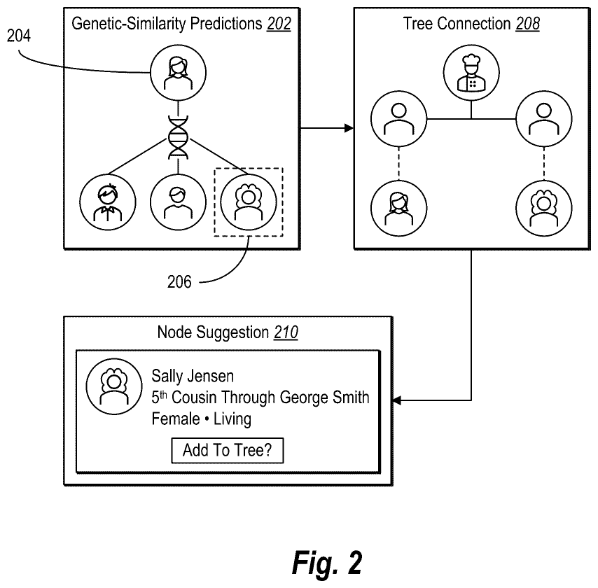

As previously mentioned, the genetic-match-suggestion system 106 provides node suggestions for a user account to add to a genealogical tree. In particular, after determining a genetic match indicating a genetic similarity between a first user account and a second user account, the genetic-match-suggestion system 106 determines there is a tree connection between the first and second account and provides a node suggestion for the first user account for a genealogical tree. FIG. 2 illustrates an example diagram of an overview of a genetic-match-suggestion system 106 determining a tree connection between a first user account and a second user account and providing a node suggestion for the first user account for a genealogical tree in accordance with one or more embodiments. Additional detail regarding the acts and processes introduced in FIG. 2 is provided thereafter with reference to subsequent figures.

As illustrated in FIG. 2, the genetic-match-suggestion system 106 generates genetic-similarity predictions 202 indicating similarities between a genetic profile of user account 204 and additional user accounts of the genealogical-data system (e.g., the genealogical-data system 104). Specifically, the genetic-match-suggestion system 106 compares the genetic profile of user account 204 with the additional genetic profiles of additional user accounts of the genealogical-data system 104 and generates genetic-similarity predictions 202 as enumerations indicating genetic similarities between user account 204 and the additional user accounts. For example, the genetic-match-suggestion system 106 generates genetic-similarity predictions 202 by generating metrics, scores, or classifications quantifying genetic similarities between a genetic profile of user account 204 and genetic profiles of additional user accounts.

In one or more embodiments, the genetic-match-suggestion system 106 generates genetic-similarity predictions 202 for user accounts that are associated with a genetic profile. More particularly, the genetic-match-suggestion system 106 analyzes genetic material (e.g., raw genotyping reads) from a third-party genetic-analysis system to generate genetic profiles for corresponding user accounts and generates genetic-similarity predictions 202 between user accounts that have overlapping genetic profiles. For example, the genetic-match-suggestion system 106 analyzes genetic profiles and identifies portions of genetic material within the genetic profiles that match and generates genetic-similarity predictions between user accounts. In some cases, through a system integration between the genetic-match-suggestion system 106 and the third-party genetic-analysis system 110, the genetic-match-suggestion system 106 associates the genetic profile from the third-party genetic-analysis system 110 with the user account for the genetic-match-suggestion system 106.

In one or more embodiments, the genetic-match-suggestion system 106 generates genetic-similarity predictions 202 by comparing genetic material (e.g., segments of DNA) in the genetic profile of the user account to genetic material (e.g., segments of DNA) in additional genetic profiles of additional user accounts. In particular, the genetic-match-suggestion system 106 generates genetic-similarity predictions 202 by comparing genetic segments, such as single nucleotide polymorphisms (SNPs) or identical-by-descent (IBD) regions. In some cases, after comparing genetic profiles, the genetic-match-suggestion system 106 generates genetic-similarity predictions 202 by measuring or comparing centimorgans (cM) indicating lengths of genetic segments and generating a metric corresponding to the cumulative cM shared between particular genetic profiles. In additional cases, after comparing the genetic profiles, the genetic-match-suggestion system 106 generates genetic-similarity predictions 202 as metrics or scores indicating shared genetic segments between the genetic profiles (e.g., cumulative cM or number of segments shared between genetic profiles).

Based on the genetic-similarity predictions, the genetic-match-suggestion system 106 determines a genetic match indicating user account 206 matches user account 204. Specifically, the genetic-match-suggestion system 106 determines a genetic match based on a genetic-similarity prediction between the user accounts. In one or more embodiments, the genetic-match-suggestion system 106 determines that a genetic-similarity prediction indicates a genetic match between user account 206 and user account 204 based on the genetic-similarity prediction satisfying a genetic-match threshold. Specifically, the genetic-match-suggestion system 106 determines a genetic match based on a number of shared genetic segments and/or on a cumulative cM shared. For example, the genetic-match-suggestion system 106 compares genetic segments shared between user accounts to generate a metric indicating a total number of shared genetic segments and/or a cumulative cM shared.

In one or more embodiments, the genetic-match-suggestion system 106 determines a segment is a shared genetic segment based on the length of shared genetic material. Specifically, the genetic-match-suggestion system 106 determines that genetic segments are a shared genetic segment if the shared length is at least threshold length (e.g., 6 cM, or other predefined lengths such as 1 cM, 10 cM, 25 cM, 50 cM, 100 cM, etc.). Moreover, the genetic-match-suggestion system 106 can determine a closer biological relationship based on the length of a shared genetic segment, as longer shared segments indicate closer biological relationships. For example, a parent-child relationship will have or feature a longer shared genetic segment (e.g., roughly 3,400 cM) than a sibling relationship (e.g., roughly 2,000 cM) or a cousin relationship (e.g., roughly 680 cM).

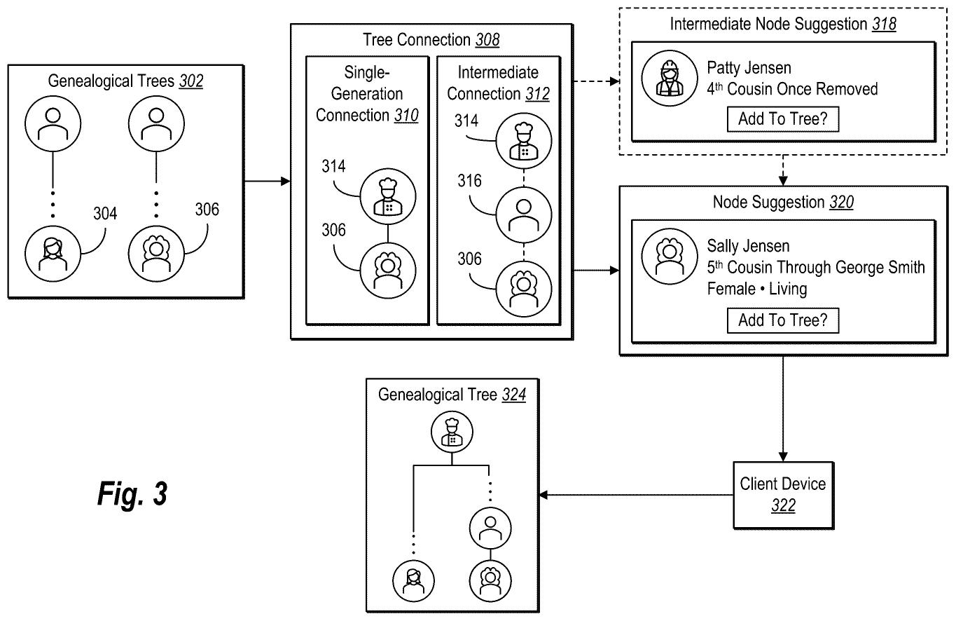

As also illustrated in FIG. 2, the genetic-match-suggestion system 106 determines there is a tree connection 208 between user account 204 and user account 206. Specifically, the genetic-match-suggestion system 106 compares a genealogical tree of user account 204 and a genealogical tree of user account 206 to determine if there is a tree connection to a shared node. For example, the genetic-match-suggestion system 106 determines there is a tree connection between user account 204 and user account 206 if there is a single-generation connection that directly connects user account 206 to a shared node with user account 204 (e.g., user account 206 has a parent-child relationship or sibling relationship with the shared node). In other examples, the genetic-match-suggestion system 106 determines there is a tree connection when user account 206 is connected to a shared node with user account 206 through an additional node or multiple additional nodes that act as intermediate connections to a shared node. Additional details regarding the genetic-match-suggestion system 106 determining a tree connection are provided below with respect to FIG. 3.

As further illustrated in FIG. 2, the genetic-match-suggestion system 106 provides a node suggestion 210 for user account 204. Specifically, the genetic-match-suggestion system 106 provides node suggestion 210 for user account 204 to add a node corresponding to user account 206. For example, the genetic-match-suggestion system 106 provides node suggestion 210 corresponding to user account 206 as a recommended node based on a genetic match to user account 204. By providing node suggestions to user accounts, the genetic-match-suggestion system 106 is able to provide valuable information to user accounts that would otherwise not have access to these genetic matches. For instance, user accounts can reach a point where they have exhausted their resources and are unable to add additional nodes to their genealogical trees. Through providing node suggestions corresponding to genetic matches, the genetic-match-suggestion system 106 facilitates connections between user accounts that can lead to additional genealogical records, genealogical stories (or additional information to add to a genealogical story), and even sharing of genealogical records between user accounts.

The genetic-match-suggestion system 106 also provides node suggestion 210 corresponding to user account 206 as a suggestion to add data to an existing node for (or corresponding to) user account 206. As an example, the genetic-match-suggestion system 106 provides node suggestion 210 corresponding to user account 206 as recommended data (e.g., genetic-match data) to add to an existing node for or corresponding to user account 206 within a genealogical tree of user account 204. Indeed, through providing both node suggestions to add data to existing nodes, the genetic-match-suggestion system 106 provides opportunities for user accounts to create genealogical trees with better historical accuracy, deeper personal insights, and improved connections.

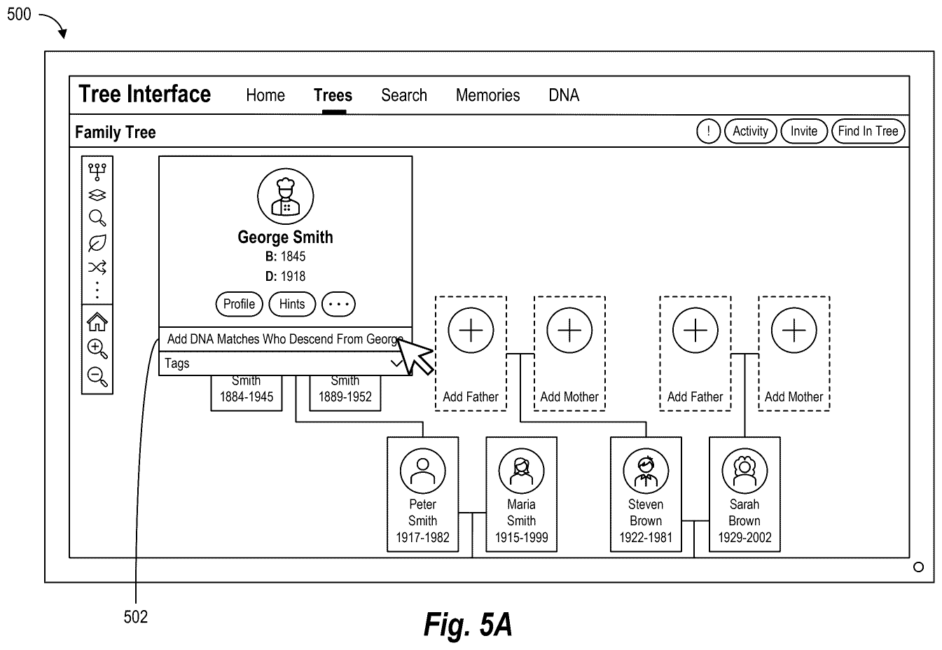

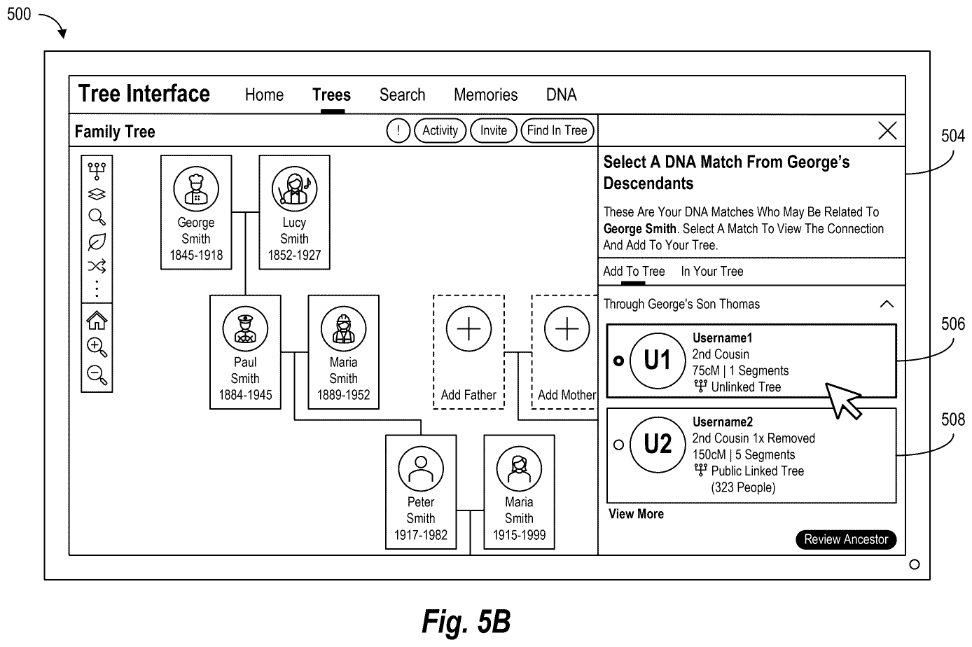

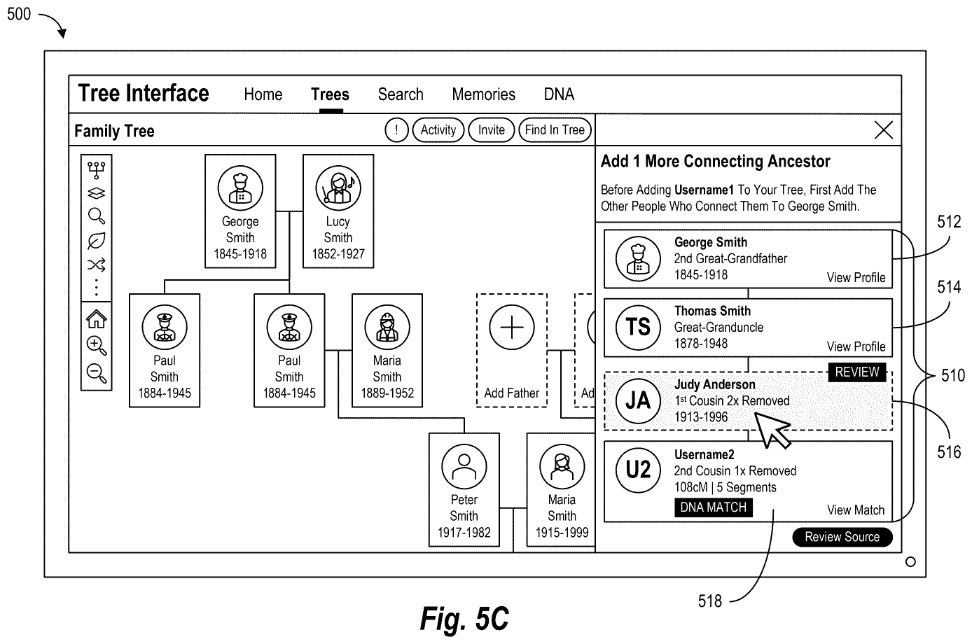

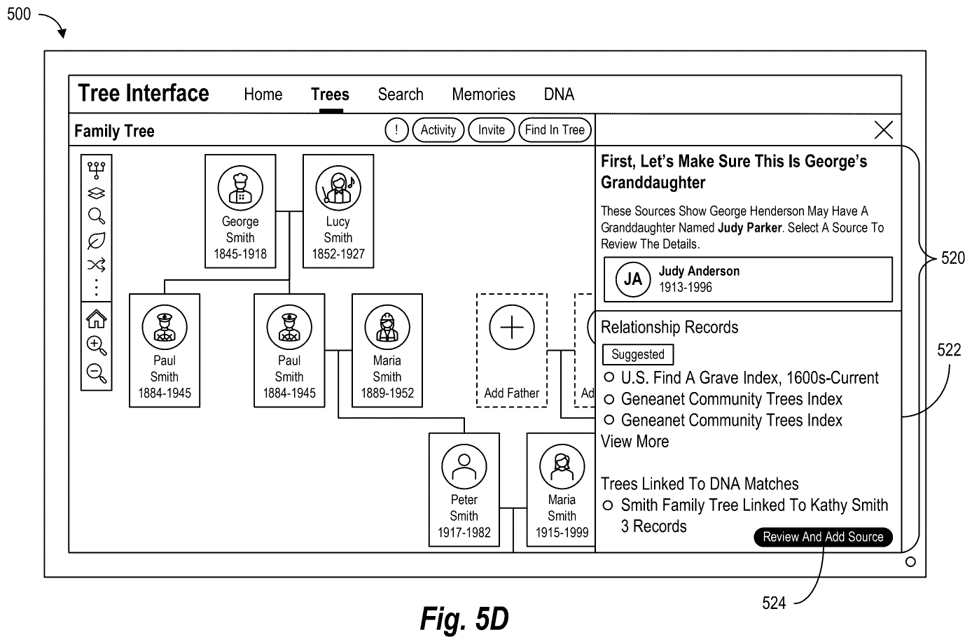

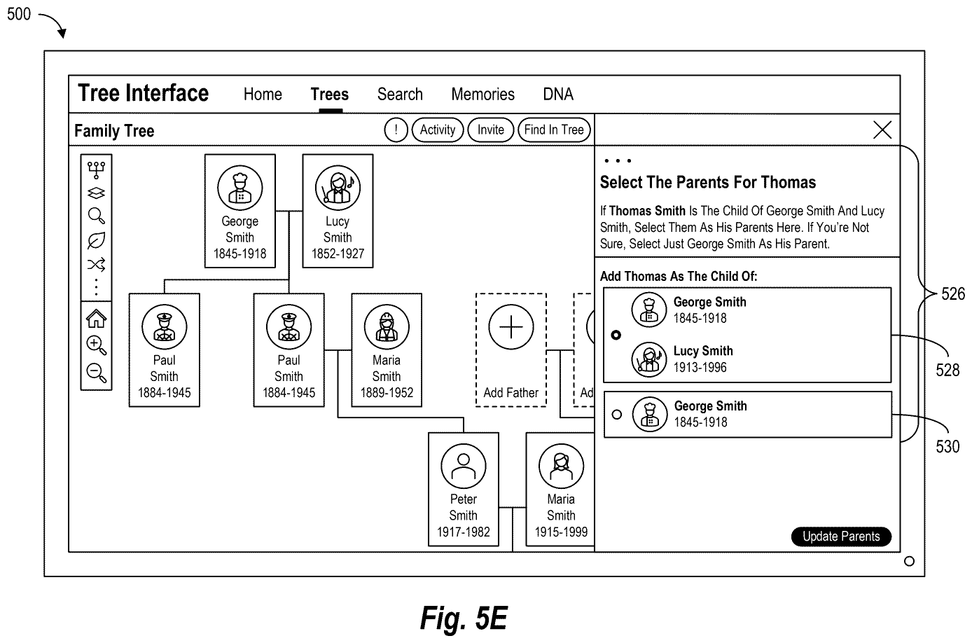

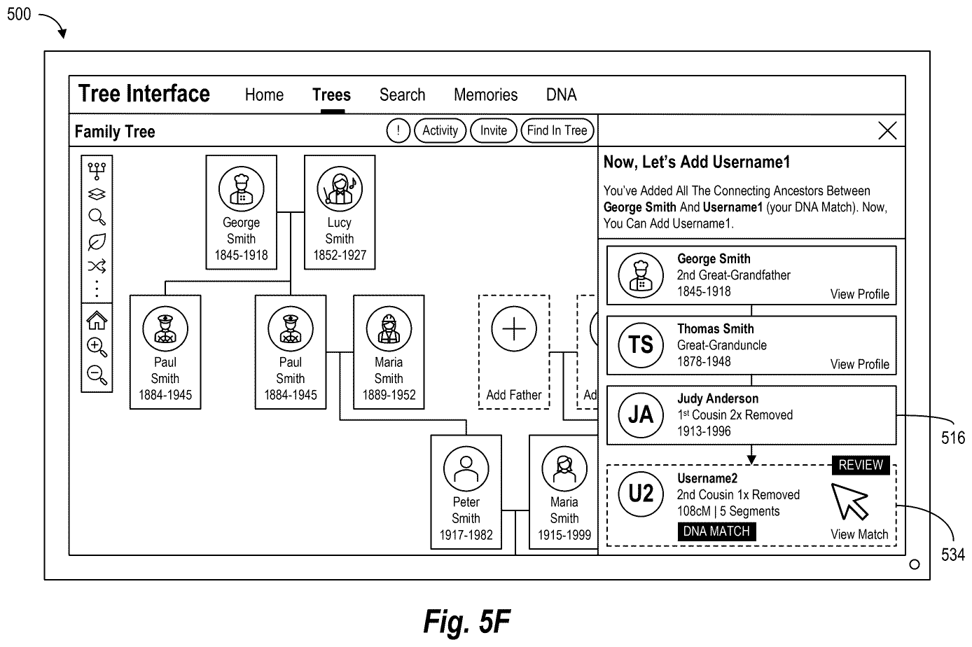

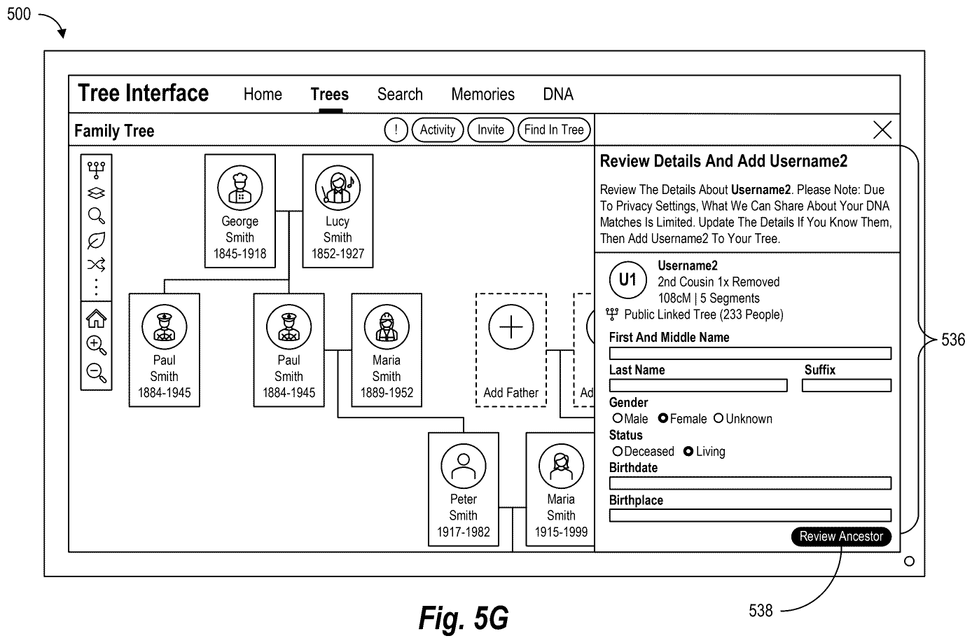

In one or more embodiments, the genetic-match-suggestion system 106 provides node suggestion 210 in response to receiving a user selection of an option to display user accounts with genetic matches. In particular, in response to the user selection of the option, the genetic-match-suggestion system 106 provides a display of genetic matches for a specified node of a genealogical tree. Based on selection of the option to display genetic matches, the genetic-match-suggestion system 106 identifies, using the genetic-similarity predictions 202, that user account 206 matches user account 204. Additional details regarding the genetic-match-suggestion system 106 providing node suggestions in response to a user selection of an option to display genetic matches for a particular node are provided below with respect to FIGS. 5A-5G.

In addition, in some embodiments, the genetic-match-suggestion system 106 provides an indication of tree connection 208 between user account 204 and user account 206 with node suggestion 210. For example, the genetic-match-suggestion system 106 provides an indication of a shared node between user account 204 and user account 206. In some cases, the genetic-match-suggestion system 106 provides an indication of the tree connection 208 (e.g., a description of the relationships) that connects user account 204 and user account 206 to the shared node. In some instances, the genetic-match-suggestion system 106 provides a visualization of the tree connection 208 and/or the shared node between user account 204 and user account 206 and the shared node. Additional details and examples regarding the genetic-match-suggestion system 106 receiving requests to display genetic matches and providing visualizations are provided below with respect to FIGS. 5A-5G.

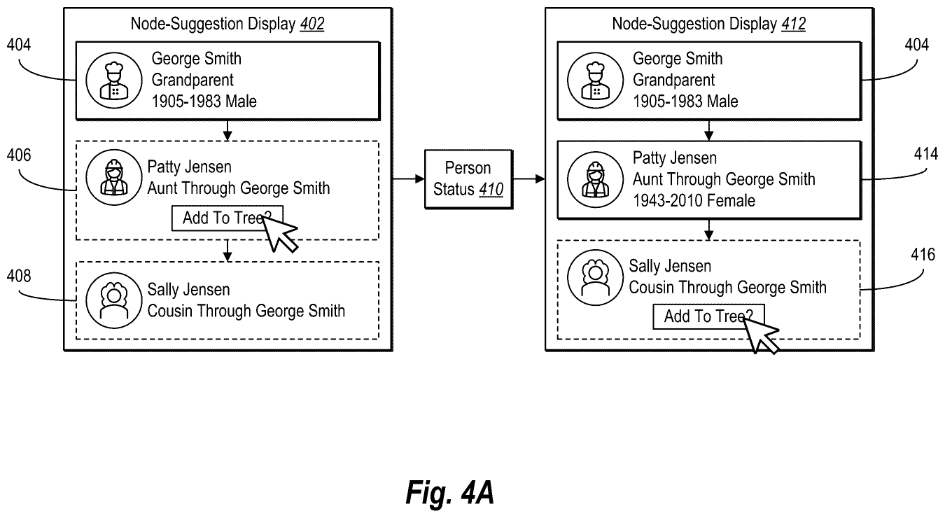

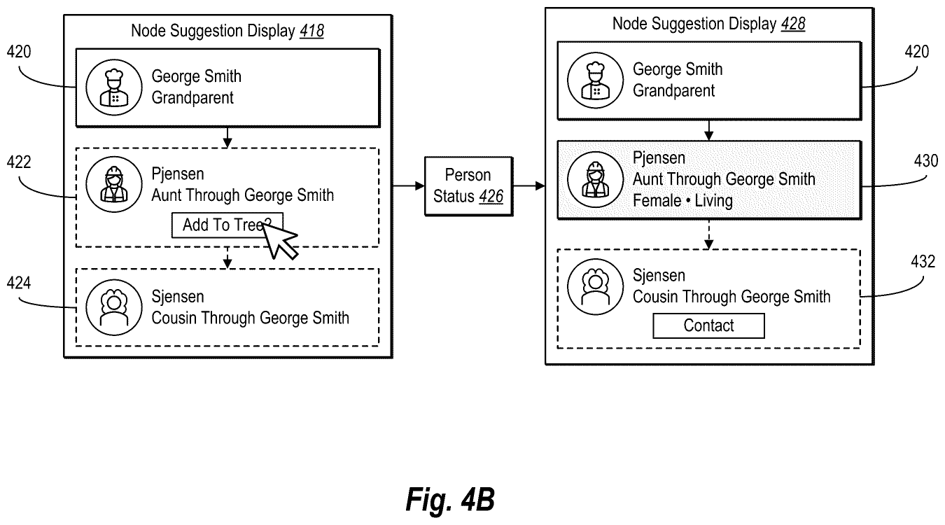

Additionally, in one or more embodiments, the genetic-match-suggestion system 106 provides additional node suggestions that are necessary to establish a tree connection between user account 204 and user account 206. In particular, the genetic-match-suggestion system 106 provides a node suggestion of an intermediate node that establishes a tree connection between user account 206 and a shared node. For instance, prior to providing node suggestion 210 to add a node for user account 206 to the genealogical tree, the genetic-match-suggestion system 106 determines that there is not a tree connection between user account 206 and the shared node. Based on determining that adding an intermediate node would establish a tree connection between user account 206 and the shared node, the genetic-match-suggestion system 106 provides a node suggestion to user account 204 to add the intermediate node to a genealogical tree. Additional details regarding the genetic-match-suggestion system 106 providing node suggestions to add intermediate nodes to a genealogical tree to establish a tree connection to a shared node are provided below with respect to FIGS. 4A-4B.

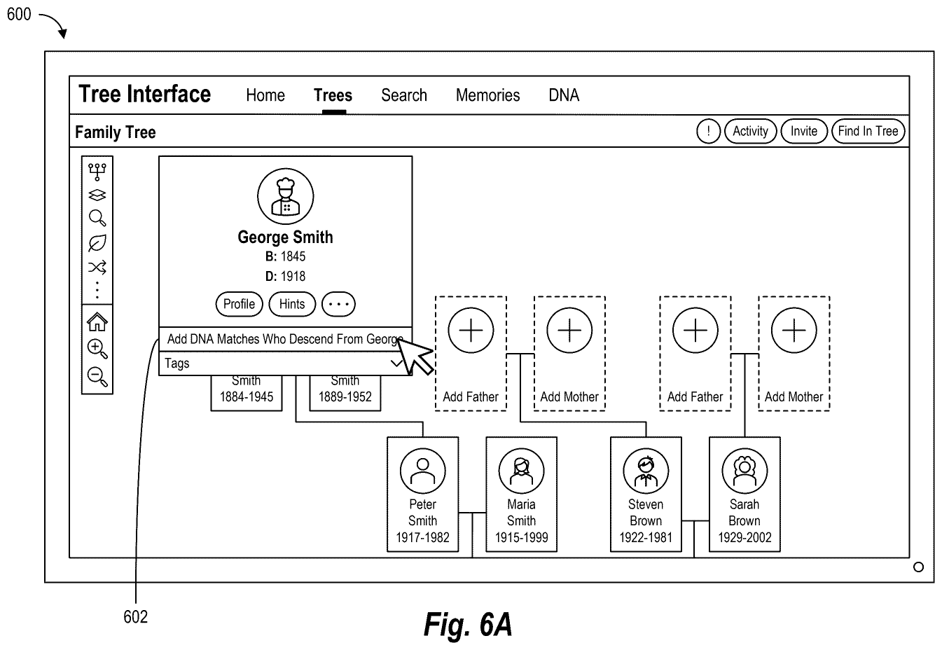

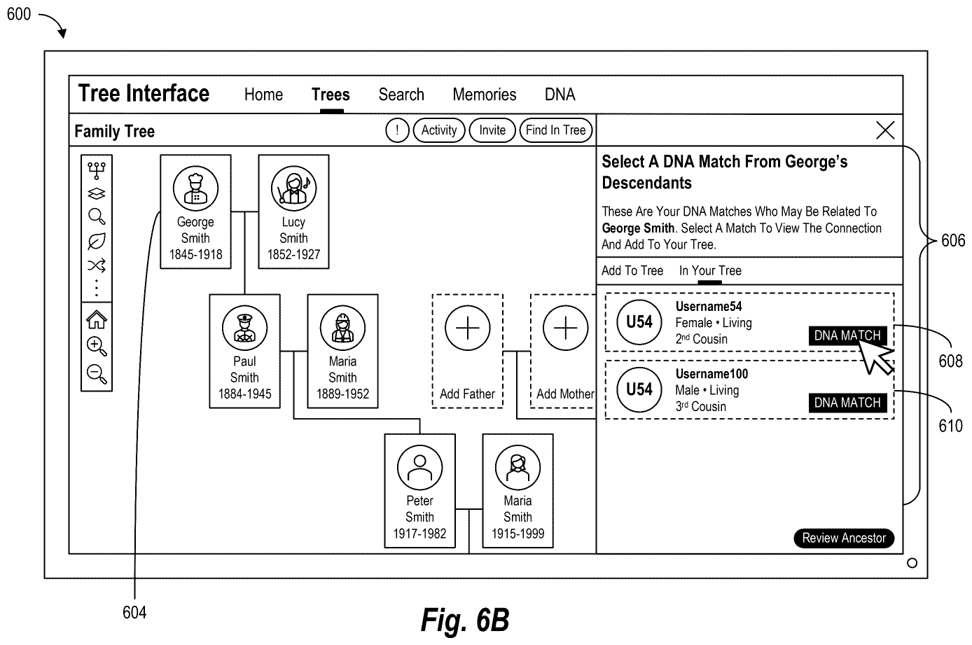

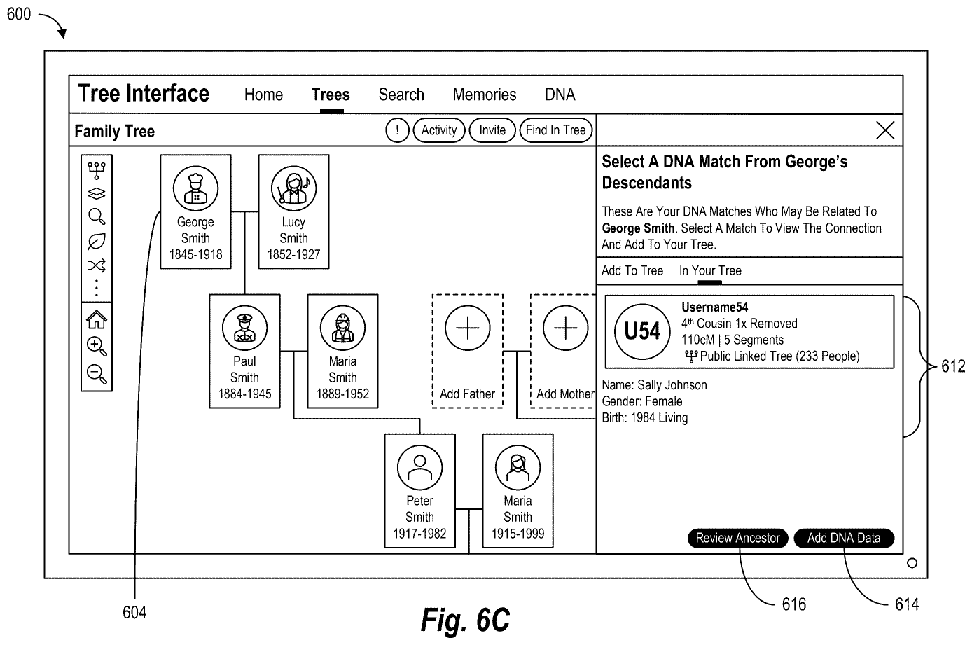

As previously mentioned, the genetic-match-suggestion system 106 can provide a node suggestion to add data or information to a node of a genealogical tree. In particular, the genetic-match-suggestion system 106 provides options to add genetic data to an existing node in a genealogical tree of user account 204. For example, based on a user interaction to add genetic data to an existing node, the genetic-match-suggestion system 106 can update the existing node with the genetic information. Additional information and details regarding the genetic-match-suggestion system 106 updating an existing node are provided below with respect to FIG. 3. In addition, example graphical user interfaces of the genetic-match-suggestion system 106 updating an existing node are provided below with respect to FIGS. 6A-6B.

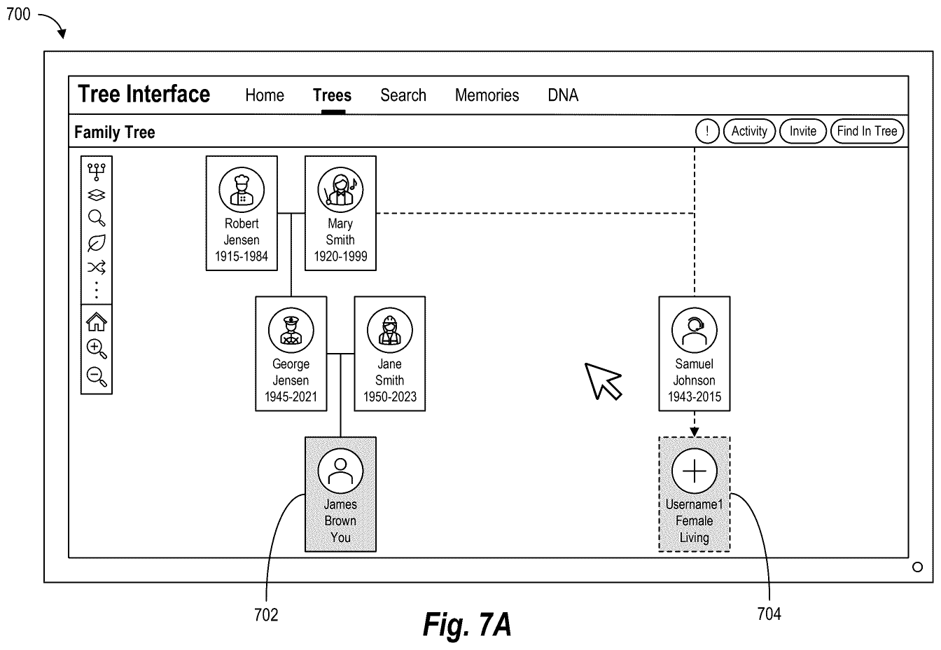

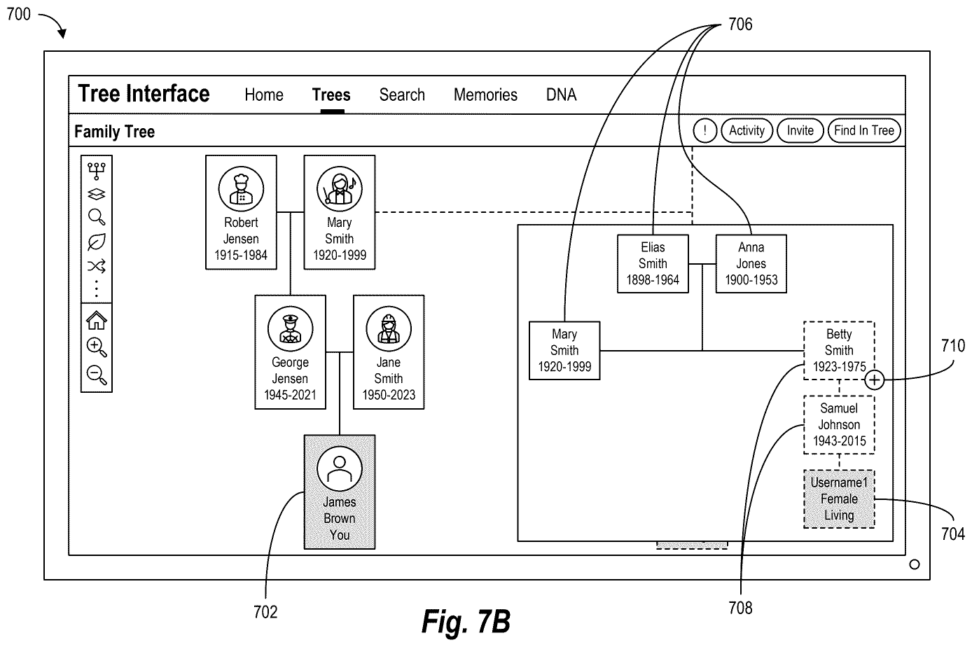

Further, in one or more embodiments, the genetic-match-suggestion system 106 can provide an indication of a node suggestion within a genealogical tree. In particular, the genetic-match-suggestion system 106 provides a visualization that indicates a location for a node suggestion in a genealogical tree. In addition, the genetic-match-suggestion system 106 can provide a visualization of a portion of a genealogical tree for a user account associated with the node suggestion indicating tree connections to a shared node. Additional details regarding the genetic-match-suggestion system 106 providing a visualization of a location of a node suggestion in a genealogical tree are provided below with respect to FIGS. 7A-7B.

As previously mentioned, in some embodiments the genetic-match-suggestion system 106 provides a node suggestion for a first user account to add a node for or corresponding to a second user account to a genealogical tree. Specifically, the genetic-match-suggestion system 106 can provide a node suggestion if there is a tree connection between the second user account and a shared node (e.g. a most-recent common ancestor of users associated with the first and second user accounts) and, based on receiving a user interaction requesting the genetic-match-suggestion system 106 add the node, adding the node to a genealogical tree through the tree connection. FIG. 3 illustrates a schematic diagram of a genetic-match-suggestion system identifying a tree connection for a suggested node and adding a node to a genealogical tree through the tree connection in accordance with one or more embodiments.

As shown in FIG. 3, the genetic-match-suggestion system 106 compares genealogical trees 302. In some embodiments, the genetic-match-suggestion system 106 compares genealogical trees 302 by comparing a genealogical tree for user account 304 to genealogical trees associated with a large-scale network of genealogical-data system 104, such as a universal genealogical tree maintained at the genealogical-data system 104 as a ground-truth source of nodes and edges defining genealogical relationships. For instance, the genetic-match-suggestion system 106 utilizes a cluster database (e.g., cluster database 914) that stores clusters of genealogical records (e.g., where each record cluster corresponds to an individual) maintained system-wide, and where the cluster database informs nodes and edges of a tree database (e.g., tree database 912) for the universal genealogical tree. The genetic-match-suggestion system 106 can thus compare the genealogical tree of user account 304 with the universal genealogical tree by comparing data of the genealogical tree with data stored in the cluster database.

For example, the genetic-match-suggestion system 106 identifies tree connection 308 that connects the genealogical tree associated with user account 304 to a genealogical tree of a user account that is not listed in the genealogical tree for user account 304. Indeed, by identifying tree connections between the genealogical tree of user account 304 and additional user accounts of the genealogical-data system 104 with genetic matches to user account 304, the genetic-match-suggestion system 106 provides unique opportunities to connect user accounts with genetic matches while maintaining a cohesive genealogical tree. In one or more embodiments, the genetic-match-suggestion system 106 compares genetic matches to genealogical trees as described in U.S. Pat. No. 11,482,306 B2, entitled GRAPHICAL USER INTERFACE DISPLAYING RELATEDNESS BASED ON SHARED DNA, filed Feb. 27, 2020, which is hereby incorporated by reference in its entirety.

In one or more embodiments, the genetic-match-suggestion system 106 compares genealogical trees 302 by comparing genealogical trees between user accounts (user accounts of the genealogical-data system 104 that have a genetic match with user account 304). More specifically, the genetic-match-suggestion system 106 compares a genealogical tree for user account 304 and a genealogical tree for user account 306 to identify if the genealogical trees have any shared nodes. The genetic-match-suggestion system 106 can identify a shared node by identifying a node in each genealogical tree that contains similar information and/or data. For example, the genetic-match-suggestion system 106 can determine that the genealogical trees for user account 304 and user account 306 have a shared node if a node includes similar information, such as name, dates (e.g., birth date, death date, marriage dates, other important dates), tree connections (e.g., connections to the same nodes), locations, or other identifying information.

In some embodiments, after comparing nodes of genealogical trees of user account 304 and user account 306, if the genetic-match-suggestion system 106 determines that some information and/or data of a node does not match, the genetic-match-suggestion system 106 can determine the nodes are a shared node if the nodes satisfy a shared-node threshold. A shared-node threshold indicates a likelihood that nodes identified in different genealogical trees are the same node (e.g., an individual associated with the node is a shared ancestor of user account 304 and user account 306). For example, the genetic-match-suggestion system 106 can generate a metric for measuring a similarity between nodes, such as a percentage of matching information between the nodes, and if the metric satisfies the shared-node threshold, the genetic-match-suggestion system 106 determines the nodes are a shared node.

As illustrated, the genetic-match-suggestion system 106 also establishes if there is a tree connection 308 between user account 304 and user account 306. Specifically, the genetic-match-suggestion system 106 determines that there is a tree connection in a genealogical tree by identifying a currently established connection between nodes of a genealogical tree or determining that adding a node (or multiple nodes) to a genealogical tree will establish a connection between nodes. For example, a tree connection could be through a shared node (e.g., shared node 314) located in both genealogical trees, and adding a node for user account 306 would establish a connection to the shared node. In addition, in some embodiments, after comparing genealogical trees of user account 304 and user account 306, the genetic-match-suggestion system 106 identifies a tree connection based on identifying that there is already a node for user account 306 in a genealogical tree for user account 304 through an established tree connection to another node of the genealogical tree for user account 304.

As mentioned, the genetic-match-suggestion system 106 determines the tree connection 308 between user account 304 and user account 306 by determining there is a tree connection between user account 306 and a shared node 314. As illustrated, the genetic-match-suggestion system 106 can determine tree connection 308 between user account 304 and user account 306 by determining whether there is a single-generation connection 310 or an intermediate connection 312 between user account 306 and shared node 314. The genetic-match-suggestion system 106 determines there is a single-generation connection 310 by identifying there can be a connection that directly connects user account 306 to shared node 314, with no intervening nodes (e.g., no generations) between user account 306 and shared node 314. For instance, the genetic-match-suggestion system 106 can determine that there is a direct biological link between user account 306 and shared node 314, so a node for user account 306 can be directly connected to shared node 314. To illustrate, nodes could have a single-generation connection if the individuals associated with the nodes have a parent-child relationship or a biological sibling relationship.

The genetic-match-suggestion system 106 determines alternatively that there is an intermediate connection 312 between user account 306 and shared node 314 by identifying a connection through an intermediate node 316. Specifically, the genetic-match-suggestion system 106 determines that shared node 314 and user account 306 can be connected in a genealogical tree for user account 304 through connections to intermediate node 316 (e.g., shared node 314 and a node for user account 306 are separated by a generation through intermediate node 316). For example, the genetic-match-suggestion system 106 determines that shared node 314 has a tree connection to intermediate node 316, and also that intermediate node 316 has a tree connection to user account 306. To illustrate an intermediate connection 312, an individual associated with shared node 314 could be a grandparent to an individual associated with user account 306.

In one or more embodiments, intermediate connection 312 can include multiple nodes between user account 306 and shared node 314. Specifically, though intermediate connection 312 is illustrated in FIG. 3 with only intermediate node 316 connecting shared node 314 and a node for user account 306, there can be several intermediate nodes between shared node 314 and a node for user account 306. For example, an individual associated with user account 306 could have a great-grandchild relationship or a great-great-grandchild relationship (or so forth going back several generations) with an individual associated with shared node 314. A s another example, user account 306 could have a staggered intermediate tree connection, where user account 306 is connected to, but is not a direct descendent of, shared node 314, such as if an individual associated with user account 306 has a great-niece/great-nephew relationship with an individual associated with shared node 314.

As previously mentioned, the genetic-match-suggestion system 106 can determine tree connection 308 based on identifying that adding one or more nodes will establish a tree connection between shared node 314 and user account 306. For example, the genetic-match-suggestion system 106 can determine that there is a potential single-generation tree connection between a node for user account 306 and shared node 314 (e.g., adding a node for user account 306 would establish a single-generation connection to shared node 314). As another example, the genetic-match-suggestion system 106 can determine there is a potential intermediate tree connection between shared node 314 and user account 306 through intermediate node 316 (e.g., adding a node for intermediate node 316 and a node for user account 306 would establish an intermediate tree connection between shared node 314 and user account 306).

As further illustrated, if there is a tree connection between user account 306 and shared node 314, the genetic-match-suggestion system 106 generates node suggestion 320 and provides node suggestion 320 on client device 322. For instance, the genetic-match-suggestion system 106 generates node suggestion 320 that prompts user account 304 to add user account 306 to a genealogical tree of user account 304. In some embodiments, the genetic-match-suggestion system 106 displays a visualization along with node suggestion 320 visualizing a tree connection between user account 306 and user account 304. In some cases, the visualization also indicates a tree connection between user account 306 and shared node 314. Examples of a visualization that indicates a tree connection between user account 306 and shared node 314 are provided below with respect to FIG. 5C, FIG. 5F, and FIGS. 7A-7B.

In one or more embodiments, the genetic-match-suggestion system 106 determines tree connection 308 by identifying a node corresponding to user account 306 in the genealogical tree for user account 304. More particularly, based on comparing genealogical trees 302, the genetic-match-suggestion system 106 determines that the genealogical tree for user account 304 has an existing node for user account 306 (e.g., already in the genealogical tree). In these instances, the genetic-match-suggestion system 106 generates node suggestion 320 to prompt the user account to update the node corresponding to user account 306. For example, the genetic-match-suggestion system 106 can provide node suggestion 320 to add data, content items, genetic information, or other information to the node corresponding to user account 306. Additional details and examples of the genetic-match-suggestion system 106 providing a node suggestion to update an existing node in a genealogical tree are provided below with respect to FIGS. 6A-6C.

As previously mentioned, in one or more embodiments, the genetic-match-suggestion system 106 determines tree connection 308 by determining that user account 306 is not currently connected to shared node 314 but determines there is a potential tree connection through intermediate node 316. In cases where there is a potential tree connection through an intermediate node, the genetic-match-suggestion system 106 provides intermediate node suggestion 318. In some cases, the genetic-match-suggestion system 106 provides intermediate node suggestion 318 to client device 322 prior to providing node suggestion 320 to establish a tree connection to shared node 314 (e.g., to avoid providing a node suggestion for a node that cannot be connected into a genealogical tree).

In one or more embodiments, the genetic-match-suggestion system 106 adds node suggestion 320 to genealogical tree 324 for user account 304 based on a user interaction with node suggestion 320. In particular, after providing the node suggestion 320 on the client device 322 associated with user account 304, the genetic-match-suggestion system 106 receives a user interaction with node suggestion 320 requesting genetic-match-suggestion system 106 add a node for user account 306 to genealogical tree 324. In addition, the genetic-match-suggestion system 106 can receive a user interaction with intermediate node suggestion 318 and add a node (such as intermediate node 316) corresponding to intermediate node suggestion 318 to genealogical tree 324, then add a node corresponding to node suggestion 320 (such as a node corresponding to user account 306) after receiving an additional user interaction with node suggestion 320.

As mentioned, the genetic-match-suggestion system 106 provides a node suggestion for an intermediate node to establish a tree connection within a genealogical tree to a shared node. Specifically, the genetic-match-suggestion system 106 provides a node suggestion to add an intermediate node to the genealogical tree prior to providing a node suggestion for a user account indicated as a genetic match. FIGS. 4A-4B illustrate a schematic diagram of a genetic-match-suggestion system providing node suggestions to add intermediate nodes to a genealogical tree to establish a tree connection to a shared node in accordance with one or more embodiments. Specifically, FIG. 4A illustrates providing a node suggestion to add an intermediate node and then providing a node suggestion for a user account indicated as a genetic match after adding the intermediate node as a deceased person node to the genealogical tree. FIG. 4B illustrates providing a node suggestion to add an intermediate node to a genealogical tree and, if the intermediate node is a living person, adding the intermediate node as a living person node and an option to contact the user account indicated as a genetic match.

As also shown in FIG. 4A, the genetic-match-suggestion system 106 provides a node-suggestion display 402 that includes node suggestion 406 to add an intermediate node to a genealogical tree prior to adding a node corresponding to a user account 408, where user account 408 is a user account that shares or is a genetic match with a user account of the genealogical tree. Specifically, when the genetic-match-suggestion system 106 determines there is not an established tree connection to a shared node (e.g. a most-recent common ancestor George Smith) but that adding an intermediate node would establish a tree connection, the genetic-match-suggestion system 106 provides node suggestion 406 with an option to add node suggestion 406 to the genealogical tree.

In addition, the genetic-match-suggestion system 106 provides node-suggestion display 402 by providing a visualization of user account 408 and a visualization of a tree connection (or potential tree connection) between shared node 404, node suggestion 406, and user account 408. Importantly, the genetic-match-suggestion system 106 does not provide an option to add user account 408 directly to the genealogical tree in node-suggestion display 402. Indeed, the genetic-match-suggestion system 106 provides options to add nodes to a genealogical tree if there is an established tree connection to a shared node within the genealogical tree (or if adding the node establishes a tree connection within the genealogical tree). In some cases, the genetic-match-suggestion system 106 does not provide options to add nodes, such as node corresponding to user account 408, directly to a genealogical tree if an intermediate node is required to establish a tree connection and there is not an established tree connection to an intermediate node (e.g., to avoid providing options to add nodes that would “float” in the genealogical tree). That is, a user may be required to build the connection between the shared node 404 and the user account 408 step-by-step to avoid an orphan node, such as user account 408, being present in the genealogical tree without edges establishing and corroborating its location within the genealogical tree. Thus the user may be guided intuitively to build a documented branch of a family tree within the genealogical tree so as to connect the user account with the user account 408.

As shown, in addition to a tree connection, the genetic-match-suggestion system 106 displays limited personal information for node suggestion 406 and user account 408 in node-suggestion display 402. In particular, the genetic-match-suggestion system 106 displays personal information in node-suggestion display 402 according to a privacy setting associated with nodes associated with user accounts of living persons. For example, user accounts of living persons may have privacy settings that limit personal information displayed for living persons to other users to gender and status (e.g., living or deceased). In some cases, the genetic-match-suggestion system 106 (or the genealogical-data system 104) allows user accounts to select user preferences indicating if they would like gender information displayed. However, for nodes associated with deceased persons, the genetic-match-suggestion system 106 can display additional personal information for deceased persons within node-suggestion display 402.

In addition, in some embodiments, the genetic-match-suggestion system 106 can display personal information in node-suggestion display 402 based on user preferences. For instance, the genetic-match-suggestion system 106 receives selections of user preferences indicating whether or not the genetic-match-suggestion system 106 can display personal information (or what information the genetic-match-suggestion system 106 can display) and displays personal information in node-suggestion display 402 accordingly.

As further illustrated, the genetic-match-suggestion system 106 determines a person status 410. Specifically, the genetic-match-suggestion system 106 determines person status 410, which indicates whether an individual associated with node suggestion 406 is a living person or a deceased person. Indeed, the genetic-match-suggestion system 106 can distinguish between living persons and deceased persons in generating and customizing interfaces to include or omit various information. For example, based on privacy settings and governing rules, the genetic-match-suggestion system 106 can exclude certain information regarding living persons that might otherwise be included for deceased persons.

For example, the genetic-match-suggestion system 106 determines person status 410 after receiving a user interaction to add a node corresponding to node suggestion 406 but prior to adding a node for node suggestion 406. Based on person status 410, the genetic-match-suggestion system 106 determines whether to add a node for node suggestion 406 as a living person node or a deceased person node. According to privacy constraints, a living person node will display limited personal or identifying information (e.g., based on government regulations), such as only displaying a gender and a status (e.g., living). A deceased person node will display additional personal or identifying information, such as birth date, death date, locations lived, historical records, content items, and connections to other deceased person nodes.

As mentioned, based on receiving a user interaction with node suggestion 406, the genetic-match-suggestion system 106 adds node 414, which corresponds to node suggestion 406. In particular, the genetic-match-suggestion system 106 adds node 414 as a branch of a genealogical tree extending from shared node 404. As shown, when the genetic-match-suggestion system 106 determines from person status 410 that node suggestion 406 corresponds to a deceased person, the genetic-match-suggestion system 106 adds node 414 as a deceased person node extending from shared node 404.

The genetic-match-suggestion system 106 also updates node-suggestion display 402 to node-suggestion display 412 to indicate that node suggestion 406 is now added to the genealogical tree as node 414, where node 414 is a deceased person node. If the genetic-match-suggestion system 106 determines from person status 410 that node suggestion 406 corresponds to a living person, then the genetic-match-suggestion system 106 adds a living person node to node-suggestion display 412. An example of the genetic-match-suggestion system 106 adding an intermediate note as a living person node is provided below with respect to FIG. 4B.

As shown, because node 414 is a deceased person node, the genetic-match-suggestion system 106 displays additional personal information that was not provided in node suggestion 406. For example, the genetic-match-suggestion system 106 adds personal or identifying information such as birth and death dates, locations, historical records, and connections/relationships to nodes of other deceased persons. In some cases, the genetic-match-suggestion system 106 displays a user account name or an abbreviated version of the full name and adds a full name to node 414 based on adding node 414 as a deceased person node.

As also illustrated, when the genetic-match-suggestion system 106 updates node-suggestion display 402 to node-suggestion display 412, the genetic-match-suggestion system 106 also provides node suggestion 416 corresponding to user account 408. In particular, node suggestion 416 includes an option within node suggestion 416 to add user account 408 to the genealogical tree based on adding node 414 to the genealogical tree as an intermediate node. For example, after adding node 414 to the genealogical tree, the genetic-match-suggestion system 106 determines there is established a tree connection within the genealogical tree to shared node 404, thus generating a place to establish a tree connection to user account 408 to shared node 404.

As previously mentioned, in one or more embodiments, the genetic-match-suggestion system 106 adds a node corresponding to a node suggestion as a living person node. In particular, if a person status indicates an individual associated with a node suggestion is a living person, the genetic-match-suggestion system 106 adds a node to a genealogical tree by adding a living person node. FIG. 4B illustrates adding a living person node to a genealogical tree as an intermediate node and an option to contact the user account indicated in a genetic match.

As shown in FIG. 4B, in one or more embodiments, when the genetic-match-suggestion system 106 identifies from person status 426 that node suggestion 422 corresponds to a living person, the genetic-match-suggestion system 106 adds an intermediate node corresponding to node suggestion 422 as living person node 430 as a branch of a genealogical tree extending from shared node 420. In particular, upon adding living person node 430, the genetic-match-suggestion system 106 updates node-suggestion display 418 to node-suggestion display 428, which indicates a change from node suggestion 422 to living person node 430. For example, as shown, the genetic-match-suggestion system 106 can provide a living person node 430 in node-suggestion display 428, which includes text, shading, borders, or other indications that the node is a living person node.

In addition, the genetic-match-suggestion system 106 provides a display 432 in node-suggestion display 428 corresponding to user account 424 with an option to contact user account 424. In some embodiments, the genetic-match-suggestion system 106 does not provide a node suggestion with an option to add a node corresponding to user account 424. Instead, the genetic-match-suggestion system 106 provides a display 432 indicating a location for user account 424 in a genealogical tree in relation to shared node 420 and living person node 430 (an intermediate node) and an option to contact user account 424. For example, by utilizing the option to contact user account 424, a user account of the genealogical tree could request information or knowledge to add nodes and/or data to the genealogical tree.

In some cases, the genetic-match-suggestion system 106 also provides an indication that node suggestion 422 is a living person in node-suggestion display 418. Specifically, the genetic-match-suggestion system 106 can display different information when node suggestion 422 and/or a display for user account 424 are associated with a living person. For example, as shown, the genetic-match-suggestion system 106 can display a user account name for node suggestion 422 and/or the display of user account 424 instead of a given name (e.g., a name as indicated on government documentation). By providing a user account name instead of a given name, the genetic-match-suggestion system 106 indicates that the person is a living person. The genetic-match-suggestion system 106 provides further indication (or confirmation) that a node suggestion corresponds to a living person when a user account requests the genetic-match-suggestion system 106 add a node corresponding to the user account to a genealogical tree, as shown in living person node 430 in node-suggestion display 428.

As previously mentioned, the genetic-match-suggestion system 106 provides a node suggestion to add a node for a second user account to a genealogical account for a first user account. In particular, the genetic-match-suggestion system 106 provides node suggestions within a genealogical-tree interface. FIGS. 5A-5G illustrate example graphical user interfaces of the genetic-match-suggestion system providing and displaying node suggestions to add to a genealogical tree in accordance with one or more embodiments.

As shown in FIG. 5A, the genetic-match-suggestion system 106 provides a genealogical-tree interface 500 that displays a genealogical tree for a user account of a genealogical-data system. For example, the genetic-match-suggestion system 106 provides displays of nodes of the genealogical tree, which also indicates tree connections between nodes within genealogical-tree interface 500. In some cases, selecting a node will bring additional information and/or options corresponding to the node, such as genealogical records associated with the node, additional details about the individual associated with the node (e.g., locations lived, important dates), content items (e.g., pictures, digital historical records), or other information stored that corresponds to the node.

As illustrated, in one or more embodiments, in response to a user selection of a node of the genealogical tree, for example where the selected node displays an indicium of being an ancestor to genetic matches of the user, the genetic-match-suggestion system 106 provides option 502 to add said user accounts that descend from the node and are genetic matches of the user account. Based on receiving a user selection of option 502, the genetic-match-suggestion system 106 can provide a list of genetic matches indicating user accounts that are genetic matches of the user account associated with the genealogical tree and are determined to be descendants of the selected node, i.e. the shared node. For example, in response to receiving a user selection of option 502, the genetic-match-suggestion system 106 can access stored (or cached) genetic-similarity predictions to identify genetic matches. As another example, the genetic-match-suggestion system 106 can access stored (or cached) user accounts that are genetic matches of the user account through the corresponding node (e.g., the node associated with option 502).

As illustrated in FIG. 5B, in response to a user selection of option 502, the genetic-match-suggestion system 106 displays window 504 that displays node suggestion 506 and node suggestion 508. In particular, node suggestion 506 and node suggestion 508 display user accounts that are genetic matches of the user account of the genealogical tree through the selected node, i.e. through the shared node. In some cases, node suggestion 506 and node suggestion 508 include selectable options that, based on a user selection, indicate a user selection to add a node corresponding to the selected node suggestion. The node suggestions 506, 508 may display a predicted relationship label, e.g. “second cousin,” “second cousin once removed,” along with genetic-match details such as a cumulative shared IBD (e.g. 17 cM) and/or a number of shared segments (e.g. 5 segments).

As illustrated in FIG. 5C, in response to a selection of node suggestion 506, the genetic-match-suggestion system 106 provides a visualization 510 that depicts a portion of the genealogical tree that includes a depiction of user account 518 corresponding to node suggestion 506. For example, visualization 510 indicates shared node 512, an established tree connection to intermediate node 514, a potential tree connection to intermediate node suggestion 516, and a depiction of user account 518 that includes a visual indication that the user account 518 is a genetic match (e.g., “DNA match”).

As shown, the genetic-match-suggestion system 106 provides intermediate node suggestion 516 as a suggestion of an intermediate node to add to the genealogical tree to establish a tree connection to shared node 512. In particular, the genetic-match-suggestion system 106 determines that there is not a node in the genealogical tree corresponding to intermediate node suggestion 516 and provides intermediate node suggestion 516. For instance, if added to the genealogical tree, intermediate node suggestion 516 establishes a tree connection within the genealogical tree from user account 518 toward the shared node 512. Further, the intermediate node suggestion 516 may include an indicium prompting a user to review the node before adding the node to the tree. In embodiments, the user may be prompted to corroborate the intermediate node suggestion 516 by reviewing one or more sources, such as potentially corroborating historical records, publicly available genealogical trees, or other sources available through a genealogical research service to confirm that the intermediate node suggestion 516 is, in fact, pertinent to the shared node 512 and/or the user account 518.

As illustrated in FIG. 5D, based on a user selection of intermediate node suggestion 516, the genetic-match-suggestion system 106 provides genealogical record 522 that is associated with shared node 512 and/or intermediate node suggestion 516. In particular, the genetic-match-suggestion system 106 provides genealogical record 522 for the user account associated with the genealogical tree to review to confirm that intermediate node suggestion 516 is connected to shared node 512 in the manner indicated in visualization 510. For example, genealogical record 522 can be a content item that includes (or depicts) historical and/or genealogical information that the genetic-match-suggestion system 106 determines may include information confirming intermediate node suggestion 516 is connected to shared node 512 in the manner indicated in visualization 510. This step advantageously improves the quality of a cluster database (e.g., cluster database 914) by ensuring that connections between nodes—e.g., between the user account 518 and the user account of the genealogical tree—are grounded in corroborating evidence, thereby reducing the confounding effects of inadvertently merged nodes that actually represent different individuals.

In addition, the genetic-match-suggestion system 106 provides option 524 to confirm that intermediate node suggestion 516 is connected to the shared node in the manner indicated in visualization 510. For example, based on receiving a selection of option 524, the genetic-match-suggestion system 106 determines that genealogical record 522 indicates intermediate node suggestion 516 is connected to shared node 512 in the manner indicated in visualization 510. In some instances, the genetic-match-suggestion system 106 provides a full representation (or replication) of all of genealogical record 522 within window 520, and a selection of option 524 confirms that genealogical record 522 indicates intermediate node suggestion 516 is connected to shared node 512 in the manner indicated in visualization 510. In other instances, genealogical record 522 in window 520 is a portion of the genealogical record or a summary of the genealogical record, such as an Al-generated summary, and a user selection of option 524 opens another window with a full depiction of a genealogical record with an additional option to confirm the genealogical record indicates that intermediate node suggestion 516 is connected to shared node 512 in the manner indicated in visualization 510.

As illustrated in FIG. 5E, in some embodiments, the genetic-match-suggestion system 106 provides options 526 to confirm parents for intermediate node 514, an additional node in the tree connection between intermediate node suggestion 516 and shared node 512. In particular, the genetic-match-suggestion system 106 provides options 526 as additional confirmation that information for intermediate node 514 is accurate. As shown, in some cases, options 526 can include option 528 to confirm that the indicated nodes are parents to intermediate node 514 and option 530 to confirm that only one node is a parent to intermediate node 514. The genetic-match-suggestion system 106 provides multiple options for parentage to account for different parentage situations, including, but not limited to, different biological parents due to remarriage, adoption, polygamy, or genetic evidence indicating that a biological parent is different from a previously established parent. Options 526 further improve the resolution and accuracy of the aforementioned cluster database by ensuring that edges between distinct nodes are corroborated by accurate evidence.

As illustrated in FIG. 5F, based on receiving a confirmation of the genealogical record 522, the genetic-match-suggestion system 106 can provide node suggestion 534 that corresponds to user account 518. Specifically, the genetic-match-suggestion system 106 provides node suggestion 534 based on adding a node corresponding to intermediate node suggestion 516 to the genealogical tree, thereby establishing a tree connection within the genealogical tree for user account 518. For example, node suggestion 534 is a selectable option that, when the genetic-match-suggestion system 106 receives a selection of the option from a client device associated with the user account of the genealogical tree, requests the genetic-match-suggestion system 106 to add a node corresponding to the user account to the genealogical tree.

As illustrated in FIG. 5G, in response to receiving a selection of node suggestion 534 to add the user account to the genealogical tree, the genetic-match-suggestion system 106 provides panel 536 to provide details for a user account associated with node suggestion 534. As previously mentioned, privacy settings limit the information that the genetic-match-suggestion system 106 can provide for some user accounts corresponding to living persons (i.e. user accounts associated with living persons who have set their profiles to “private” rather than “public”), and the genetic-match-suggestion system 106 provides panel 536 for a user account to add details to a node for the node representing the user account in the genealogical tree. For example, a user account can add known information for the node for the user account for the genetic-match-suggestion system 106 (or the genealogical-data system 104) to save in the node in the genealogical tree.

As shown, the genetic-match-suggestion system 106 can also provide option 538. Upon receiving a user selection of option 538, the genetic-match-suggestion system 106 adds a node corresponding to a user account associated with node suggestion 534 to the genealogical tree. In particular, the genetic-match-suggestion system 106 can add a node corresponding to a user account associated with node suggestion 534 with information provided in panel 536 to the genealogical tree. For example, if a user adds information in panel 536, the genetic-match-suggestion system 106 includes that information when adding a node based on node suggestion 534 to the genealogical tree.

As previously mentioned, the genetic-match-suggestion system 106 can add information to an existing node in a genealogical tree. Specifically, the genetic-match-suggestion system 106 can update an existing node in a genealogical tree with genealogical data based on identifying a genetic match indicating the existing node matches the user account of the genealogical tree. FIGS. 6A-6C illustrate example graphical user interfaces of the genetic-match-suggestion system 106 updating existing nodes of a genealogical tree with genetic data in accordance with one or more embodiments.

As illustrated in FIG. 6A, the genetic-match-suggestion system 106 provides, within a genealogical-tree interface 600 and in response to a user selection of a node of a genealogical tree, option 602 to add genetic data to an existing node in the genealogical tree. Specifically, the genetic-match-suggestion system 106 identifies genetic matches that correspond to existing nodes within the genealogical tree that descend from the selected node, i.e. from a shared common ancestor. For example, in response to receiving a user selection of option 602, the genetic-match-suggestion system 106 can utilize genetic-similarity predictions to identify genetic matches that correspond to existing nodes. Further, because the genetic-match-suggestion system 106 identifies nodes that descend from the selected node, the genetic-match-suggestion system 106 can also treat the selected node as a shared node.

As illustrated in FIG. 6B, in response to a user selection of option 602, the genetic-match-suggestion system 106 provides window 606 within genealogical-tree interface 600 that includes existing node 608 and existing node 610. The genetic-match-suggestion system 106 provides existing node 608 and existing node 610 within window 606 based on determining genetic matches that indicate that users associated with existing node 608 and existing node 610 are genetic matches of the user account of the genealogical tree and descendants of selected node 604, i.e. the shared common ancestor. For example, the genetic-match-suggestion system 106 determines that the existing node 608 and existing node 610 share a certain (e.g. above a predetermined threshold number) number of cumulative centimorgans of shared genetic material, e.g. IBD, and/or that they share a number of segments (e.g., a threshold number of segments) with selected node 604.

As illustrated in FIG. 6B, the genetic-match-suggestion system 106 receives a user selection of existing node 608. Continuing to FIG. 6C, based on the selection of existing node 608, the genetic-match-suggestion system 106 updates window 606 to include window 612 that indicates information and genetic data for existing node 608. For example, the genetic-match-suggestion system 106 provides the genetic data for selected node 604 along with stored information for the existing node 608. Based on a user selection of option 614, the genetic-match-suggestion system 106 updates the existing node with the genetic data indicated in window 612. In addition, as illustrated, the genetic-match-suggestion system 106 also provides option 616 to review and/or update the existing node. For instance, the genetic-match-suggestion system 106 can receive user interactions adding or deleting information from the existing node 608.

In one or more embodiments, the genetic-match-suggestion system 106 provides an indication of where a suggested node fits in a genealogical tree. Specifically, the genetic-match-suggestion system 106 provides an indication in a genealogical tree that indicates where a suggested node fits in the genealogical tree. FIGS. 7A-7B illustrates example graphical user interfaces for providing a preview of a location of a node suggestion within a genealogical tree in accordance with one or more embodiments. In particular, FIG. 7A illustrates an example graphical user interface for providing a location of a node suggestion within a genealogical tree of a user account. FIG. 7B illustrates an example graphical user interface for displaying a portion of a genealogical tree for the node suggestion.

As illustrated in FIG. 7A, the genetic-match-suggestion system 106 provides or displays a genealogical tree for a user account 702 within a genealogical-tree interface 700 that includes an indication of a location within the genealogical tree for node suggestion 704. Specifically, the genetic-match-suggestion system 106 provides node suggestion 704 with indications of tree connections to a shared node within the genealogical tree for user account 702. For example, as shown, the genetic-match-suggestion system 106 can provide dashed lines that indicate where node suggestion 704 would connect to the genealogical tree for user account 702.

As illustrated in FIG. 7B, the genetic-match-suggestion system 106 can also display a portion of a genealogical tree for the node suggestion. For example, the genetic-match-suggestion system 106 provides a display that indicates intermediate nodes 708 with the corresponding tree connections to node suggestion 704 and shared nodes 706 that are connected within the genealogical tree of user account 702 and the genealogical tree for node suggestion 704. In some cases, in response to a user interaction with node suggestion 704, the genetic-match-suggestion system 106 provides a display of a portion of a genealogical tree to node suggestion 704 (e.g., in a window of genealogical-tree interface 700 or by generating an interface for the genealogical tree of node suggestion).

As indicated, the genealogical tree can visualize or include renderings of indications of intermediate nodes 708. Specifically, the genetic-match-suggestion system 106 provides indications that intermediate nodes 708 would provide tree connections to shared nodes 706, but tree connections are not yet established to shared nodes 706 within the genealogical tree of user account 702. For example, as shown, the genetic-match-suggestion system 106 displays intermediate nodes 708 in a different manner than nodes that are established in the genealogical tree of user account 702, including shared nodes 706.

In addition, as shown, the genetic-match-suggestion system 106 also includes option 710 to add an intermediate node of intermediate nodes 708. In particular, the genetic-match-suggestion system 106 provides option 710 to an intermediate node that is directly connected with a single-generation connection to a shared node of shared nodes 706. For example, as shown, the genetic-match-suggestion system 106 provides an option to add “Betty Smith,” who has a single-generation connection to “Mary Smith” (e.g., a sibling connection), “Elias Smith” (e.g., a parent-child connection), and “Anna Jones” (e.g., a parent-child connection). By providing option 710 with the intermediate node, the genetic-match-suggestion system 106 can establish a tree connection within the genealogical tree of user account 702 to further add the other intermediate node of intermediate nodes 708 (“Samuel Johnson”) and then add a node for node suggestion 704.

In one or more embodiments, the genetic-match-suggestion system 106 displays a portion of a genealogical tree based on user permissions. The genetic-match-suggestion system 106 can thus adapt the presentation or generation of tree interfaces according to user account settings (e.g., where some or all of a genealogical tree of a user account is public and/or private). For example, the genetic-match-suggestion system 106 can modify a genealogical tree of a user account based on automated incorporations of data from other genealogical trees and/or genetic data. Alternatively, the genetic-match-suggestion system 106 can provide notifications or suggestions (e.g., as views of other genealogical trees or genetic data indications) to modify a genealogical tree of a user account but can wait to apply such modifications until selection or verification by the user account. The genetic-match-suggestion system 106 can thus adapt the presentation of tree connections, genetic matches, and/or other genealogical tree data on a node-by-node basis, depending on settings for user accounts corresponding to (e.g., whose profile is represented by) respective nodes.

Specifically, if user preferences for a user account associated with node suggestion 704 indicate that other user accounts may view their genealogical tree, then the genetic-match-suggestion system 106 can provide a visualization of a portion of the genealogical tree encompassed by the window including the node suggestion 704 and its connecting nodes. The genetic-match-suggestion system 106 can, in some cases, provide the tree portion to include both the node suggestion 704 as well as a node for the user account 702 to visualize where in the tree the node suggestion 704 fits relative to the user account 702. In some cases, a user preference of the user account for node suggestion 704 may indicate that any users may view a portion of the genealogical tree for the user account. In other cases, a user preference of the user account for node suggestion 704 may indicate that only certain users may view a portion of the genealogical tree for the user account (thus preventing other user accounts from viewing or accessing data from that portion of the genealogical tree), such as individuals within a threshold number of edges to the user account, as indicated by the user account's genealogical tree and/or a universal genealogical tree of the genealogical-data system 104. For example, a user preference may indicate that only users with a tree connection can view the genealogical tree. As another example, user preferences may indicate that only selected users can view the genealogical tree.

FIG. 8 is a block diagram of an architecture of an example computing server 800 (e.g., the server(s) 102), in accordance with some embodiments. In the embodiment shown in FIG. 8, the computing server 800 includes a genealogy data store 802, a genetic data store 804, an individual profile store 806, a sample pre-processing engine 808, a phasing engine 810, an identity by descent (IBD) estimation engine 812, a community assignment engine 814, an IBD network data store 816, a reference panel sample store 818, an ethnicity estimation engine 820, a front-end interface 822, and a tree management engine 824. The functions of the computing server 800 may be distributed among the elements in a different manner than described. In various embodiments, the computing server 800 may include different components and fewer or additional components. Each of the various data stores may be a single storage device, a server controlling multiple storage devices, or a distributed network that is accessible through multiple nodes (e.g., a cloud storage system).

The computing server 800 stores various data of different individuals, including genetic data, genealogy data, and survey response data. The computing server 800 processes the genetic data of individuals (e.g., genetic profiles) to identify shared identity-by-descent (IBD) segments between individuals. The genealogy data and survey response data may be part of user account profile data. The amount and type of user account profile data stored for each individual may vary based on the information of an individual, which determined or received from the individual through creating an account and profile at a system (e.g., the genealogical-data system 104) operated by the computing server 800, and through updating the profile, family tree, and social network at the system, in some cases linking a profile with genetic data. The computing server 800 may also include answers to survey questions regarding various traits of individuals, such as phenotypes, characteristics, preferences, habits, lifestyle, and environment for user accounts.

The genealogy data store 802 may store genealogy data, including various types of data that are related to tracing family relatives of individuals. Examples of genealogy data include names (first, last, middle, suffixes), gender, birth locations, date of birth, date of death, marriage information, spouse's information kinships, family history, dates and places for life events (e.g., birth and death), other vital data, and the like. In some instances, family history can take the form of a pedigree of an individual (e.g., the recorded relationships in the family). The family tree information associated with an individual may include one or more specified nodes. Each node in the family tree represents the individual, an ancestor of the individual who might have passed down genetic material to the individual, and the individual's other relatives including siblings, cousins, and offspring in some cases. Genealogy data may also include connections and relationships among user accounts of the computing server 800.

In addition to user account data, genealogy data may also take other forms that are obtained from various sources such as public records and third-party data collectors. For example, genealogical records from public sources include birth records, marriage records, death records, census records, court records, probate records, adoption records, and/or obituary records. Likewise, genealogy data may include data from one or more family trees of an individual, the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, and/or a motor vehicle database.

Furthermore, the genealogy data store 802 may also include relationship information inferred from the genetic data stored in the genetic data store 804 and information received from the individuals. For example, the relationship information may indicate which individuals are genetically related, how they are related, how many generations back they share common ancestors, lengths and locations of IBD segments shared, which genetic communities an individual is a part of, variants carried by the individual, and the like.

The computing server 800 maintains genetic datasets of individuals in the genetic data store 804. A genetic dataset of an individual may be a digital dataset of nucleotide data (e.g., SNP data) and corresponding metadata. A genetic dataset may contain data on the whole or portions of an individual's genome. The genetic data store 804 may store a pointer to a location associated with the genealogy data store 802 associated with the individual. A genetic dataset may take different forms. In some embodiments, a genetic dataset may take the form of a base pair sequence of the sequencing result of an individual. A base pair sequence dataset may include the whole genome of the individual (e.g., obtained from a whole-genome sequencing) or some parts of the genome (e.g., genetic loci of interest).

In another embodiment, a genetic dataset may take the form of sequences of genetic markers. Examples of genetic markers may include target SNP loci (e.g., allele sites) filtered from the sequencing results. A SNP locus that is single base pair long may also be referred to a SNP site. A SNP locus may be associated with a unique identifier. The genetic dataset may be in a form of diploid data that includes a sequencing of genotypes, such as genotypes at the target SNP loci, or the whole base pair sequence that includes genotypes at known SNP loci and other base pair sites that are not commonly associated with known SNPs. The diploid dataset may be referred to as a genotype dataset or a genotype sequence. Genotype may have a different meaning in various contexts. In one context, an individual's genotype may refer to a collection of diploid alleles of an individual. In other contexts, a genotype may be a pair of alleles present on two chromosomes for an individual at a given genetic marker such as a SNP site.

Genotype data for a SNP site may include a pair of alleles. The pair of alleles may be homozygous (e.g., A-A or G-G) or heterozygous (e.g., A-T, C-T). Instead of storing the actual nucleotides, the genetic data store 804 may store genetic data that are converted to bits. For a given SNP site, oftentimes only two nucleotide alleles (instead of all 4) are observed. As such, a 2-bit number may represent a SNP site. For example, 00 may represent homozygous first alleles, 11 may represent homozygous second alleles, and 01 or 10 may represent heterozygous alleles. A separate library may store what nucleotide corresponds to the first allele and what nucleotide corresponds to the second allele at a given SNP site.

A diploid dataset may also be phased into two sets of haploid data, one corresponding to a first parent side and another corresponding to a second parent side. The phased datasets may be referred to as haplotype datasets or haplotype sequences. Similar to genotype, haplotype may have a different meaning in various contexts. In one context, a haplotype may also refer to a collection of alleles that corresponds to a genetic segment. In other contexts, a haplotype may refer to a specific allele at a SNP site. For example, a sequence of haplotypes may refer to a sequence of alleles of an individual that are inherited from a parent.

The individual profile store 806 stores profiles and related metadata associated with various individuals appeared in the computing server 800. A computing server 800 may use unique individual identifiers to identify various user accounts and other non-user accounts that might appear in other data sources such as ancestors or historical persons who appear in any family tree or genealogy database. A unique individual identifier may be a hash of certain identification information of an individual, such as a user account's account name, user account's name, date of birth, location of birth, or any suitable combination of the information. The profile data related to an individual may be stored as metadata associated with an individual's profile. For example, the unique individual identifier and the metadata may be stored as a key-value pair using the unique individual identifier as a key.

An individual's profile data may include various kinds of information related to the individual. The metadata about the individual may include one or more pointers associating genetic datasets such as genotype and phased haplotype data of the individual that are saved in the genetic data store 804. The metadata about the individual may also be individual information related to family trees and pedigree datasets that include the individual. The profile data may further include declarative information about the user account that was authorized by the user account to be shared and may also include information inferred by the computing server 800. Other examples of information stored in a user account profile may include biographic, demographic, and other types of descriptive information such as work experience, educational history, gender, hobbies, or preferences, and/or location. In some embodiments, the user account profile data may also include one or more photos of the user accounts and photos of relatives (e.g., ancestors) of the user accounts that are uploaded by the user accounts. A user account may authorize the computing server 800 to analyze one or more photos to extract information, such as the user account's or relative's appearance traits (e.g., blue eyes, curved hair, etc.), from the photos. The appearance traits and other information extracted from the photos may also be saved in the profile store. In some cases, the computing server may allow user accounts to upload many different photos of the user accounts, their relatives, and even friends. User account profile data may also be obtained from other suitable sources, including historical records (e.g., records related to an ancestor), medical records, military records, photographs, other records indicating one or more traits, and other recorded data.

For example, the computing server 800 may present various survey questions to its user accounts from time to time. The responses to the survey questions may be stored at individual profile store 806. The survey questions may be related to various aspects of the user accounts and the user account families. Some survey questions may be related to user account phenotypes, while other questions may be related to environmental factors of the user accounts.

Survey questions may concern health or disease-related phenotypes, such as questions related to the presence or absence of genetic diseases or disorders, inheritable diseases or disorders, or other common diseases or disorders that have a family history as one of the risk factors, questions regarding any diagnosis of increased risk of any diseases or disorders, and questions concerning wellness-related issues such as a family history of obesity, and/or family history of causes of death. The diseases identified by the survey questions may be related to single-gene diseases or disorders that are caused by a single-nucleotide variant, an insertion, or a deletion. The diseases identified by the survey questions may also be multifactorial inheritance disorders that may be caused by a combination of environmental factors and genes. Examples of multifactorial inheritance disorders may include heart disease, Alzheimer's disease, diabetes, cancer, and obesity. The computing server 800 may obtain data on a user account's disease-related phenotypes from survey questions about the health history of the user account and her family and also from health records uploaded by the user account.

Survey questions also may be related to other types of phenotypes such as appearance traits of the user accounts. A survey regarding appearance traits and characteristics may include questions related to eye color, iris pattern, freckles, chin types, finger length, dimple chin, earlobe types, hair color, hair curl, skin pigmentation, susceptibility to skin bum, bitter taste, male baldness, baldness pattern, presence of unibrow, presence of wisdom teeth, height, and weight. A survey regarding other traits also may include questions related to user account taste and smell such as the ability to taste bitterness, asparagus smell, and/or cilantro aversion. A survey regarding traits may further include questions related to user account body conditions such as lactose tolerance, caffeine consumption, malaria resistance, norovirus resistance, muscle performance, alcohol flush, etc. Other survey questions regarding a person's physiological or psychological traits may include vitamin traits and sensory traits such as the ability to sense an asparagus metabolite. Traits may also be collected from historical records, electronic health records and electronic medical records.

The computing server 800 also may present various survey questions related to the environmental factors of user accounts. In this context, an environmental factor may be a factor that is not directly connected to the genetics of user accounts. Environmental factors may include user account preferences, habits, and lifestyles. For example, a survey regarding user account preferences may include questions related to things and activities that user accounts like or dislike, such as types of music a user account enjoys, dancing preference, party-going preference, certain sports that a user account plays, video game preferences, etc. Other questions may be related to diet preferences such as liking or disliking a certain type of food (e.g., ice cream, egg). A survey related to habits and lifestyle may include questions regarding smoking habits, alcohol consumption and frequency, daily exercise duration, sleeping habits (e.g., morning person versus night person), sleeping cycles and problems, hobbies, and travel preferences. Additional environmental factors may include diet amount (calories, macronutrients), physical fitness abilities (e.g., stretching, flexibility, heart rate recovery), family type (adopted family or not, has siblings or not, lived with extended family during childhood), property and item ownership (has home or rents, has a smartphone or does not, has a car or does not).

Surveys also may be related to other environmental factors such as geographical, social-economic, or cultural factors. Geographical questions may include questions related to the birth location, family migration history, town, or city of current or past residence. Social-economic questions may be related to education level, income, occupations, and/or self-identified demographic groups. Questions related to culture may concern native language, language spoken at home, customs, and/or dietary practices. Other questions related to cultural and behavioral questions are also possible.

For any survey questions asked, the computing server 800 may also ask an individual the same or similar questions regarding the traits and environmental factors of the ancestors, family members, other relatives or friends of the individual. For example, the computing server 800 may ask about the native language of the user account and the native languages of parents and grandparents. The computing server 800 may also ask about the health history of family members.

In addition to storing the survey data in the individual profile store 806, the computing server 800 may store some responses that correspond to data related to genealogical and genetics respectively to genealogy data store 802 and genetic data store 804.

The user account profile data, photos of user accounts, survey response data, the genetic data, and the genealogy data may be subject to the privacy and authorization setting of the user accounts to specify any data related to the user accounts that can be accessed, stored, obtained, or otherwise used. For example, when presented with a survey question, a user account may select to answer or skip the question. The computing server 800 may present user accounts from time-to-time information regarding user accounts' selection of the extent of information and data shared. The computing server 800 also may maintain and enforce one or more privacy settings for user accounts in connection with the access of the user account profile data, photos, genetic data, and other sensitive data. For example, the user account may pre-authorize the access to the data and may change the setting as wished. The privacy settings also may allow a user account to specify (e.g., by opting out, by not opting in) whether the computing server 800 may receive, collect, log, or store particular data associated with the user account for any purpose. A user account may restrict her data at various levels. For example, on one level, the data may not be accessed by the computing server 800 for purposes other than displaying the data in the user account's own profile. On another level, the user account may authorize anonymization of her data and participate in studies and research conducted by the computing server 800 such as a large-scale genetic study. On yet another level, the user account may tum some portions of her genealogy data public to allow the user account to be discovered by other user accounts (e.g., potential relatives) and be connected to one or more family trees. Access or sharing of any information or data in the computing server 800 may also be subject to one or more similar privacy policies. Data and content objects in the computing server 800 may also be associated with different levels of restriction. The computing server 800 may also provide various notification features to inform and remind user accounts of their privacy and access settings. For example, when privacy settings for a data entry allow a particular user account or other entities to access the data, the data may be described as being “visible,” “public,” or other suitable labels, contrary to a “private” label.

In some cases, the computing server 800 may have a heightened privacy protection on certain types of data and data related to certain vulnerable groups. In some cases, the heightened privacy settings may strictly prohibit the use, analysis, and sharing of data related to a certain vulnerable group. In other cases, the heightened privacy settings may specify that data subject to those settings require prior approval for access, publication, or other use. In some cases, the computing server 800 may provide the heightened privacy as a default setting for certain types of data, such as genetic data or any data that the user account marks as sensitive. The user account may opt in to sharing of those data or change the default privacy settings. In other cases, the heightened privacy settings may apply across the board for all data of certain groups of user accounts. For example, if computing server 800 determines that the user account is a minor or has recognized that a picture of a minor is uploaded, the computing server 800 may designate all profile data associated with the minor as sensitive. In those cases, the computing server 800 may have one or more extra steps in seeking and confirming any sharing or use of the sensitive data.

The sample pre-processing engine 808 receives and pre-processes data received from various sources to change the data into a format used by the computing server 800. For genealogy data, the sample pre-processing engine 808 may receive data from an individual. To collect the user account data (e.g., genealogical and survey data), the computing server 800 may cause an interactive user account interface on a client device to display interface elements in which user accounts can provide genealogy data and survey data. Additional data may be obtained from scans of public records. The data may be manually provided or automatically extracted via, for example, optical character recognition (OCR) performed on census records, town or government records, or any other item of printed or online material. Some records may be obtained by digitalizing written records such as older census records, birth certificates, and/or death certificates.

The sample pre-processing engine 808 may also receive raw data from a genetic data extraction service server. A genetic data extraction service server may perform laboratory analysis of biological samples of user accounts and generate sequencing results in the form of digital data. The sample pre-processing engine 808 may receive the raw genetic datasets from the genetic data extraction service server. Most of the mutations that are passed down to descendants are related to single-nucleotide polymorphism (SNP). SNP is a substitution of a single nucleotide that occurs at a specific position in the genome. The sample pre-processing engine 808 may convert the raw base pair sequence into a sequence of genotypes of target SNP sites. Alternatively, the pre-processing of this conversion may be performed by the genetic data extraction service server. The sample pre-processing engine 808 identifies autosomal SNPs in an individual's genetic dataset. In some embodiments, the SNPs may be autosomal SNPs. In some embodiments, 700,000 SNPs may be identified in an individual's data and may be stored in genetic data store 804. Alternatively, in some embodiments, a genetic dataset may include at least 10,000 SNP sites. In another embodiment, a genetic dataset may include at least 100,000 SNP sites. In yet another embodiment, a genetic dataset may include at least 300,000 SNP sites. In yet another embodiment, a genetic dataset may include at least 1,000,000 SNP sites. The sample pre-processing engine 808 may also convert the nucleotides into bits. The identified SNPs, in bits or in other suitable formats, may be provided to the phasing engine 810 which phases the individual's diploid genotypes to generate a pair of haplotypes for each user account.

The phasing engine 810 phases diploid genetic dataset into a pair of haploid genetic datasets and may perform imputation of SNP values at certain sites whose alleles are missing. An individual's haplotype may refer to a collection of alleles (e.g., a sequence of alleles) that are inherited from a parent.

Phasing may include a process of determining the assignment of alleles (particularly heterozygous alleles) to chromosomes. Owing to sequencing conditions and other constraints, a sequencing result often includes data regarding a pair of alleles at a given SNP locus of a pair of chromosomes but may not be able to distinguish which allele belongs to which specific chromosome. The phasing engine 810 uses a genotype phasing algorithm to assign one allele to a first chromosome and another allele to another chromosome. The genotype phasing algorithm may be developed based on an assumption of linkage disequilibrium (LD), which states that haplotype in the form of a sequence of alleles tends to cluster together. The phasing engine 810 is configured to generate phased sequences that are also commonly observed in many other samples. Put differently, haplotype sequences of different individuals tend to cluster together. A haplotype-cluster model may be generated to determine the probability distribution of a haplotype that includes a sequence of alleles. The haplotype-cluster model may be trained based on labeled data that includes known phased haplotypes from a trio (parents and a child). A trio is used as a training sample because the correct phasing of the child is almost certain by comparing the child's genotypes to the parent's genetic datasets. The haplotype-cluster model may be generated iteratively along with the phasing process with a large number of unphased genotype datasets. The haplotype-cluster model may also be used to impute one or more missing data.

By way of example, the phasing engine 810 may use a directed acyclic graph model such as a hidden Markov model (HMM) to perform the phasing of a target genotype dataset. The directed acyclic graph may include multiple levels, each level having multiple nodes representing different possibilities of haplotype clusters. An emission probability of a node, which may represent the probability of having a particular haplotype cluster given an observation of the genotypes may be determined based on the probability distribution of the haplotype-cluster model. A transition probability from one node to another may be initially assigned to a non-zero value and be adjusted as the directed acyclic graph model and the haplotype-cluster model are trained. Various paths are possible in traversing different levels of the directed acyclic graph model. The phasing engine 810 determines a statistically likely path, such as the most probable path or a probable path that is at least more likely than 95% of other possible paths, based on the transition probabilities and the emission probabilities. A suitable dynamic programming algorithm such as the Viterbi algorithm may be used to determine the path. The determined path may represent the phasing result. U.S. Pat. No. 10,679,729, entitled “Haplotype Phasing Models,” granted on Jun. 9, 2020, describes example embodiments of haplotype phasing. Other example phasing embodiments are described in U.S. Patent Application Publication No. US 2021/0034647, entitled “Clustering of Matched Segments to Determine Linkage of Dataset in a Database,” published on Feb. 4, 2021.

The IBD estimation engine 812 estimates the amount of shared genetic segments between a pair of individuals based on phased genotype data (e.g., haplotype datasets) that are stored in the genetic data store 804. IBD segments may be segments identified in a pair of individuals that are putatively determined to be inherited from a common ancestor. The IBD estimation engine 812 retrieves a pair of haplotype datasets for each individual. The IBD estimation engine 812 may divide each haplotype dataset sequence into a plurality of windows. Each window may include a fixed number of SNP sites (e.g., about 100 SNP sites). The IBD estimation engine 812 identifies one or more seed windows in which the alleles at all SNP sites in at least one of the phased haplotypes between two individuals are identical. The IBD estimation engine 812 may expand the match from the seed windows to nearby windows until the matched windows reach the end of a chromosome or until a homozygous mismatch is found, which indicates the mismatch is not attributable to potential errors in phasing or imputation. The IBD estimation engine 812 determines the total length of matched segments, which may also be referred to as IBD segments. The length may be measured in the genetic distance in the unit of centimorgans (cM). A unit of centimorgan may be a genetic length. For example, two genomic positions that are one cM apart may have a 1% chance during each meiosis of experiencing a recombination event between the two positions. The computing server 800 may save data regarding individual pairs who share a length of IBD segments exceeding a predetermined threshold (e.g., 6 cM) , in a suitable data store such as in the genealogy data store 802. U.S. Pat. No. 10,114,922, entitled “Identifying Ancestral Relationships Using a Continuous stream of Input,” granted on Oct. 30, 2018, and U.S. Pat. No. 10,720,229, entitled “Reducing Error in Predicted Genetic Relationships,” granted on Jul. 21, 2020, describe example embodiments of IBD estimation.

Typically, individuals who are closely related share a relatively large number of IBD segments, and the IBD segments tend to have longer lengths (individually or in aggregate across one or more chromosomes). In contrast, individuals who are more distantly related share relatively fewer IBD segments, and these segments tend to be shorter (individually or in aggregate across one or more chromosomes). For example, while close family members often share upwards of 71 cM of IBD (e.g., third cousins), more distantly related individuals may share less than 12 cM of IBD. The extent of relatedness in terms of IBD segments between two individuals may be referred to as IBD affinity. For example, the IBD affinity may be measured in terms of the length of IBD segments shared between two individuals.

Community assignment engine 814 assigns individuals to one or more genetic communities based on the genetic data of the individuals. A genetic community may correspond to an ethnic origin or a group of people descended from a common ancestor. The granularity of genetic community classification may vary depending on embodiments and methods used to assign communities. For example, in some embodiments, the communities may be African, Asian, European, etc. In another embodiment, the European community may be divided into Irish, German, Swedes, etc. In yet another embodiment, the Irish may be further divided into Irish in Ireland, Irish immigrated to America in 1800, Irish immigrated to America in 1902, etc. The community classification may also depend on whether a population is admixed or unadmixed. For an admixed population, the classification may further be divided based on different ethnic origins in a geographical region.

Community assignment engine 814 may assign individuals to one or more genetic communities based on their genetic datasets using machine learning models trained by unsupervised learning or supervised learning. In an unsupervised approach, the community assignment engine 814 may generate data representing a partially connected undirected graph. In this approach, the community assignment engine 814 represents individuals as nodes. Some nodes are connected by edges whose weights are based on IBD affinity between two individuals represented by the nodes. For example, if the total length of two individuals' shared IBD segments does not exceed a predetermined threshold, the nodes are not connected. The edges connecting two nodes are associated with weights that are measured based on the IBD affinities. The undirected graph may be referred to as an IBD network. The community assignment engine 814 uses clustering techniques such as modularity measurement (e.g., the Louvain method) to classify nodes into different clusters in the IBD network. Each cluster may represent a community. The community assignment engine 814 may also determine sub-clusters, which represent sub-communities. The computing server 800 saves the data representing the IBD network and clusters in the IBD network data store 816. U.S. Pat. No. 10,223,498, entitled “Discovering Population Structure from Patterns of Identity-By-Descent,” granted on Mar. 5, 2019, describes example embodiments of community detection and assignment.

The community assignment engine 814 may also assign communities using supervised techniques. For example, genetic datasets of known genetic communities (e.g., individuals with confirmed ethnic origins) may be used as training sets that have labels of the genetic communities. Supervised machine learning classifiers, such as logistic regressions, support vector machines, random forest classifiers, and neural networks may be trained using the training set with labels. A trained classifier may distinguish binary or multiple classes. For example, a binary classifier may be trained for each community of interest to determine whether a target individual's genetic dataset belongs or does not belong to the community of interest. A multi-class classifier such as a neural network may also be trained to determine whether the target individual's genetic dataset most likely belongs to one of several possible genetic communities.

Reference panel sample store 818 stores reference panel samples for different genetic communities. A reference panel sample is a genetic data of an individual whose genetic data is the most representative of a genetic community. The genetic data of individuals with the typical alleles of a genetic community may serve as reference panel samples. For example, some alleles of genes may be over-represented (e.g., being highly common) in a genetic community. Some genetic datasets include alleles that are commonly present among members of the community. Reference panel samples may be used to train various machine learning models in classifying whether a target genetic dataset belongs to a community, determining the ethnic composition of an individual, and determining the accuracy of any genetic data analysis, such as by computing a posterior probability of a classification result from a classifier.

A reference panel sample may be identified in different ways. In some embodiments, an unsupervised approach in community detection may apply the clustering algorithm recursively for each identified cluster until the sub-clusters contain a number of nodes that are smaller than a threshold (e.g., contains fewer than 1000 nodes). For example, the community assignment engine 814 may construct a full IBD network that includes a set of individuals represented by nodes and generate communities using clustering techniques. The community assignment engine 814 may randomly sample a subset of nodes to generate a sampled IBD network. The community assignment engine 814 may recursively apply clustering techniques to generate communities in the sampled IBD network. The sampling and clustering may be repeated for different randomly generated sampled IBD networks for various runs. Nodes that are consistently assigned to the same genetic community when sampled in various runs may be classified as a reference panel sample. The community assignment engine 814 may measure the consistency in terms of a predetermined threshold. For example, if a node is classified to the same community 95% (or another suitable threshold) of the times whenever the node is sampled, the genetic dataset corresponding to the individual represented by the node may be regarded as a reference panel sample. Additionally, or alternatively, the community assignment engine 814 may select N most consistently assigned nodes as a reference panel for the community.

Other ways to generate reference panel samples are also possible. For example, the computing server 800 may collect a set of samples and gradually filter and refine the samples until high-quality reference panel samples are selected. For example, a candidate reference panel sample may be selected from an individual whose recent ancestors are born at a certain birthplace. The computing server 800 may also draw sequence data from the Human Genome Diversity Project (HGDP). Various candidates may be manually screened based on their family trees, relatives' birth location, and other quality control. Principal component analysis may be used to create clusters of genetic data of the candidates. Each cluster may represent an ethnicity. The predictions of the ethnicity of those candidates may be compared to the ethnicity information provided by the candidates to perform further screening.

The ethnicity estimation engine 820 estimates the ethnicity composition of a genetic dataset of a target individual. The genetic datasets used by the ethnicity estimation engine 820 may be genotype datasets or haplotype datasets. For example, the ethnicity estimation engine 820 estimates the ancestral origins (e.g., ethnicity) based on the individual's genotypes or haplotypes at the SNP sites. To take a simple example of three ancestral populations corresponding to African, European and Native American, an admixed user account may have nonzero estimated ethnicity proportions for all three ancestral populations, with an estimate such as [0.05, 0.65, 0.30], indicating that the user account's genome is 5% attributable to African ancestry, 65% attributable to European ancestry and 30% attributable to Native American ancestry. The ethnicity estimation engine 820 generates the ethnic composition estimate and stores the estimated ethnicities in a data store of computing server 800 with a pointer in association with a particular user account.

In some embodiments, the ethnicity estimation engine 820 divides a target genetic dataset into a plurality of windows (e.g., about 1000 windows). Each window includes a small number of SNPs (e.g., 300 SNPs). The ethnicity estimation engine 820 may use a directed acyclic graph model to determine the ethnic composition of the target genetic dataset. The directed acyclic graph may represent a trellis of an inter-window hidden Markov model (HMM). The graph includes a sequence of a plurality of node groups. Each node group, representing a window, includes a plurality of nodes. The nodes represent different possibilities of labels of genetic communities (e.g., ethnicities) for the window. A node may be labeled with one or more ethnic labels. For example, a level includes a first node with a first label representing the likelihood that the window of SNP sites belongs to a first ethnicity and a second node with a second label representing the likelihood that the window of SNPs belongs to a second ethnicity. Each level includes multiple nodes so that there are many possible paths to traverse the directed acyclic graph.

The nodes and edges in the directed acyclic graph may be associated with different emission probabilities and transition probabilities. An emission probability associated with a node represents the likelihood that the window belongs to the ethnicity labeling the node given the observation of SNPs in the window. The ethnicity estimation engine 820 determines the emission probabilities by comparing SNPs in the window corresponding to the target genetic dataset to corresponding SNPs in the windows in various reference panel samples of different genetic communities stored in the reference panel sample store 818. The transition probability between two nodes represents the likelihood of transition from one node to another across two levels. The ethnicity estimation engine 820 determines a statistically likely path, such as the most probable path or a probable path that is at least more likely than 95% of other possible paths, based on the transition probabilities and the emission probabilities. A suitable dynamic programming algorithm such as the Viterbi algorithm or the forward-backward algorithm may be used to determine the path. After the path is determined, the ethnicity estimation engine 820 determines the ethnic composition of the target genetic dataset by determining the label compositions of the nodes that are included in the determined path. U.S. Pat. No. 10,558,930, entitled “Local Genetic Ethnicity Determination System,” granted on Feb. 11, 2020, and U.S. Pat. No. 10,692,587, granted on Jun. 23, 2020, entitled “Global Ancestry Determination System” describe different example embodiments of ethnicity estimation.

The front-end interface 822 displays various results determined by the computing server 800. The results and data may include the IBD affinity between a user account and another individual, the community assignment of the user account, the ethnicity estimation of the user account, phenotype prediction and evaluation, genealogy data search, family tree and pedigree, relative profile and other information. The front-end interface 822 may allow user accounts to manage their profile and data trees (e.g., family trees). The user accounts may view various public family trees stored in the computing server 800 and search for individuals and their genealogy data via the front-end interface 822. The computing server 800 may suggest or allow the user account to manually review and select potentially related individuals (e.g., relatives, ancestors, close family members) to add to the user account's data tree. The front-end interface 822 may be a graphical user account interface (GUI) that displays various information and graphical elements. The front-end interface 822 may take different forms. In one case, the front-end interface 822 may be a software application that can be displayed on an electronic device such as a computer or a smartphone. The software application may be developed by the entity controlling the computing server 800 and be downloaded and installed on the client device 112. In another case, the front-end interface 822 may take the form of a webpage interface of the computing server 800 that allows user accounts to access their family tree and genetic analysis results through web browsers. In yet another case, the front-end interface 822 may provide an application program interface (API).

The tree management engine 824 performs computations and other processes related to user accounts' management of their data trees such as family trees. The tree management engine 824 may allow a user account to build a data tree from scratch or to link the user account to existing data trees. In some embodiments, the tree management engine 824 may suggest a connection between a target individual and a family tree that exists in the family tree database by identifying potential family trees for the target individual and identifying one or more most probable positions in a potential family tree. A user account (target individual) may wish to identify family trees to which he or she may potentially belong. Linking a user account to a family tree or building a family may be performed automatically, manually, or using techniques with a combination of both. In an embodiment of an automatic tree matching, the tree management engine 824 may receive a genetic dataset from the target individual as input and search related individuals that are IBO-related to the target individual. The tree management engine 824 may identify common ancestors. Each common ancestor may be common to the target individual and one of the related individuals. The tree management engine 824 may in turn output potential family trees to which the target individual may belong by retrieving family trees that include a common ancestor and an individual who is IBO-related to the target individual. The tree management engine 824 may further identify one or more probable positions in one of the potential family trees based on information associated with matched genetic data between the target individual and DNA test takers in the potential family trees through one or more machine learning models or other heuristic algorithms. For example, the tree management engine 824 may try putting the target individual in various possible locations in the family tree and determine the highest probability position(s) based on the genetic datasets of the target individual and other DNA test takers in the family tree and based on genealogy data available to the tree management engine 824. The tree management engine 824 may provide one or more family trees from which the target individual may select. For a suggested family tree, the tree management engine 824 may also provide information on how the target individual is related to other individuals in the tree. In a manual tree building, a user account may browse through public family trees and public individual entries in the genealogy data store 802 and individual profile store 806 to look for potential relatives that can be added to the user account's family tree. The tree management engine 824 may automatically search, rank, and suggest individuals for the user account conduct manual reviews as the user account makes progress in the front-end interface 822 in building the family tree.

As used herein, “pedigree” and “family tree” may be interchangeable and may refer to a family tree chart or pedigree chart that shows, diagrammatically, family information, such as family history information, including parentage, offspring, spouses, siblings, or otherwise for any suitable number of generations and/or people, and/or data pertaining to persons represented in the chart. U.S. Pat. No. 11,429,615, entitled “Linking Individual Datasets to a Database,” granted on Aug. 30, 2022, describes example embodiments of how an individual may be linked to existing family trees.

The components of the genetic-match-suggestion system 106 can include software, hardware, or both. For example, the components of the genetic-match-suggestion system 106 can include one or more instructions stored on a computer-readable storage medium and executable by processors of one or more computing devices. When executed by one or more processors, the computer-executable instructions of the genetic-match-suggestion system 106 can cause a computing device to perform the methods described herein. Alternatively, the components of the genetic-match-suggestion system 106 can comprise hardware, such as a special purpose processing device to perform a certain function or group of functions. Additionally, or alternatively, the components of the genetic-match-suggestion system 106 can include a combination of computer-executable instructions and hardware.

Furthermore, the components of the genetic-match-suggestion system 106 performing the functions described herein may, for example, be implemented as part of a stand-alone application, as a module of an application, as a plug-in for applications including content management applications, as a library function or functions that may be called by other applications, and/or as a cloud-computing model. Thus, the components of the genetic-match-suggestion system 106 may be implemented as part of a stand-alone application on a personal computing device or a mobile device.

FIG. 9 illustrates a genealogical-data system 900 (e.g., the genealogical-data system 104) interfacing with a genealogical database 902 in accordance with one or more embodiments. For certain genealogical databases, the genealogical-data system 900 identifies groups of user nodes or records in the format of a genealogical tree or records connected by biological and other family relationships as “tree data.” The genealogical-data system 900 can thus search and process tree data stored in a genealogical database 902 (which includes a tree database 912 and a cluster database 914) to execute tasks and perform functions as described herein.

For the genealogical database 902, the genealogical-data system 900 may receive genealogical data (e.g., data records and/or genealogical data objects) for building tree data from a source selected from a ground-truth genealogical tree generated from genealogical records and trees of user accounts within the genealogical data system 900, from the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, and a motor vehicle database. Additionally, genealogical data can be user-generated. Genealogical data may also include data from a cluster database 914 derived from records and user data.

Some embodiments of the genetic-match-suggestion system 106 relate to modifying a cluster database 914 based on a user query and/or other interaction with the genetic-match-suggestion system 106. In some instances, the genealogical-data system 900 (or the genetic-match-suggestion system 106) determines and/or modifies a node connection for an individual represented by or resolved to a cluster within the cluster database 914. Indeed, the genetic-match-suggestion system 106 can analyze, add, remove, and/or modify genealogical content items organized into clusters within the cluster database 914 based on relatedness corresponding to a common individual. The genetic-match-suggestion system 106 can also access, modify, and analyze genealogical trees within the tree database 912 by, for example, adding nodes, removing nodes, and/or modifying nodes based genealogical content items (and their relationships to individuals) stored within the cluster database 914.

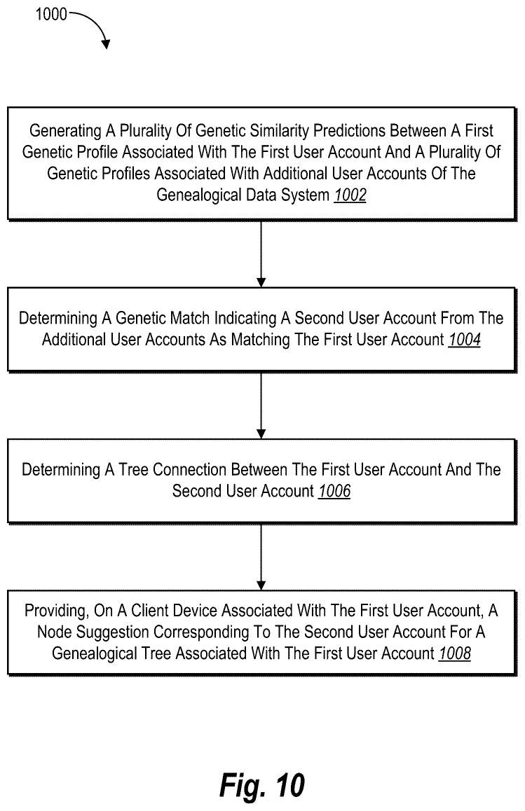

As seen in FIG. 10, the genealogical-data system 900 includes a genealogical database 902, which may include a tree database 912 and a cluster database 914. The tree database 912 may be configured to facilitate the generation, storage, and collation of family trees for a plurality of users, with trees comprising nodes and edges therebetween. Data and records, such as images, may be associated with individual nodes of the trees in the tree database 912. Tree person data, including data such as names, relationships, dates, events, and other metadata may be provided by the tree database 912 to the genealogical data system 900. The cluster database 914 may include one or more clusters comprising resolved entities, where tree persons (nodes) in different trees in the tree database 912 are associated together in a cluster after determination that the tree persons correspond to a same person.

As a user expands their family tree, e.g. by tagging a previously unknown person in an image using the suggestions provided by the ancestor-identification system and adding the now-identified person to their tree as a new node, the tree database 912 may be modified as the user's family tree is expanded, and the cluster database 914 may be modified to include the new node in the pertinent cluster. Further, the genetic-match-suggestion system 106 can attach a conversation with a user account (e.g., a query received from the user account and a response the genetic-match-suggestion system 106 generates, and/or a series of queries and corresponding responses) to a cluster within the cluster database 914 and/or a node within the tree database 912 to utilize as a ground-truth genealogical tree for future operations within the genealogical data system 900. For example, the genetic-match-suggestion system 106 can extract or otherwise pull contextual data from the user account or conversations with the user account and attach the context to a node or a cluster of the cluster database 914 to utilize as a ground-truth genealogical tree for future operations within the genealogical-data system 900 and/or the genetic-match-suggestion system 106.

FIGS. 1-9, the corresponding text, and the examples provide a number of different methods, systems, devices, and non-transitory computer-readable media of the genetic-match-suggestion system. In addition to the foregoing, one or more embodiments can also be described in terms of flowcharts comprising acts for accomplishing a particular result, as shown in FIG. 10. FIG. 10 may be performed with more or fewer acts. Further, the acts may be performed in differing orders. Additionally, the acts described herein may be repeated or performed in parallel with one another or parallel with different instances of the same or similar acts.

As mentioned, FIG. 10 illustrates a flowchart of a series of acts 1000 for determining a genetic match indicating a second user account matches a first user account and providing a suggestion to the first user account to add the second user account as a node of a genealogical tree. in accordance with one or more embodiments. While FIG. 10 illustrates acts according to one embodiment, alternative embodiments may omit, add to, reorder, and/or modify any of the acts shown in FIG. 10. The acts of FIG. 10 can be performed as part of a method. Alternatively, a non-transitory computer-readable medium can comprise instructions that, when executed by one or more processors, cause a computing device to perform the acts of FIG. 10. In some embodiments, a system can perform the acts of FIG. 10.

As shown in FIG. 10, the series of acts 1000 includes an act 1002 of generating a plurality of genetic-similarity predictions between a first genetic profile associated with the first user account and a plurality of genetic profiles associated with additional user accounts of the genealogical-data system, an act 1004 of determining a genetic match indicating a second user account from the additional user accounts as matching the first user account, an act 1006 of determining a tree connection between the first user account and the second user account, and an act 1008 of providing, on a client device associated with the first user account, a node suggestion corresponding to the second user account for a genealogical tree associated with the first user account.

In particular, the act 1002 can include generating, for a first user account of a genealogical-data system, a plurality of genetic-similarity predictions between a first genetic profile associated with the first user account and a plurality of genetic profiles associated with additional user accounts of the genealogical-data system, the act 1004 can include determining, from the plurality of genetic-similarity predictions for the first user account, a genetic match indicating a second user account from the additional user accounts as matching the first user account, the act 1006 can include based on determining the genetic match, determining a tree connection between the first user account and the second user account, and the act 1008 can include based on determining the tree connection between the first user account and the second user account, providing, on a client device associated with the first user account, a node suggestion corresponding to the second user account for a genealogical tree associated with the first user account.

For example, in one or more embodiments, the series of acts 1000 includes receiving, from the client device associated with the first user account, a user interaction with the node suggestion indicating an instruction to add a node for the second user account to the genealogical tree and based on receiving the user interaction with the node suggestion, adding the node to the genealogical tree associated with the first user account.

In addition, in one or more embodiments, the series of acts 1000 includes determining the tree connection between the first user account and the second user account further comprises comparing the genealogical tree associated with the first user account and a second genealogical tree associated with the second user account to identify a shared node. Also, in one or more embodiments, the series of acts 1000 includes determining that there is a single-generation connection within the second genealogical tree between the second user account and the shared node and providing the node suggestion with an indication of the single-generation connection between the shared node and the second user account. Further, in one or more embodiments, the series of acts 1000 includes determining that the shared node is connected within the second genealogical tree to the second user account through an intermediate node and providing the node suggestion with an indication that the shared node is connected to the second user account through the intermediate node. Moreover, in one or more embodiments, the series of acts 1000 includes receiving a user interaction to add the intermediate node to the genealogical tree associated with the first user account, based on receiving the user interaction to add the intermediate node to the genealogical tree, adding the intermediate node to the genealogical tree; receiving a user interaction with the node suggestion to add a node for the second user account to the genealogical tree, and based on receiving the user interaction to add the node for the second user account to the genealogical tree and further based on adding the intermediate node to the genealogical tree, adding the node for the second user account to the genealogical tree.

Also, in one or more embodiments, the series of acts 1000 includes based on receiving a user interaction with the node suggestion to add a node for the second user account to the genealogical tree, providing a genealogical record on the client device, receiving, from the client device, a user interaction confirming that the genealogical record is associated with a shared node, and adding the node for the second user account to the genealogical tree based on receiving the user interaction confirming that the genealogical record is associated with the shared node.

Further, in one or more embodiments, the series of acts 1000 includes providing the node suggestion further comprises determining that the tree connection between the first user account and the second user account comprises an intermediate node that corresponds to a user account of a living person; based on determining that the tree connection between the first user account and the second user account comprises an intermediate node that corresponds to the user account of the living person, providing the node suggestion comprising a suggestion to add the intermediate node to the genealogical tree as a living person node without including personal information of the living person; and providing, on the client device associated with the first user account, an option to communicate with the second user account.

Moreover, in one or more embodiments, the series of acts 1000 includes wherein providing the node suggestion further comprises providing the node suggestion with an indication of a tree connection between the node suggestion corresponding to the second user account and a shared node of the genealogical tree and a second genealogical tree of the second user account.

Additionally, in one or more embodiments, the series of acts 1000 includes receiving, from the client device, a user interaction with an option to display user accounts having genetic matches to the shared node of the genealogical tree of the first user account and providing the node suggestion and the indication of the shared node in response to receiving the user interaction to display suggested user accounts.

Moreover, in one or more embodiments, the series of acts 1000 includes receiving, from the client device associated with the first user account, a user interaction with the node suggestion and based on receiving the user interaction with the node suggestion, adding a node for the second user account to the genealogical tree as a branch of the genealogical tree extending from the shared node. In addition, in one or more embodiments, the series of acts 1000 includes adding the node for the second user account to the genealogical tree by adding a username, a gender, and a person status associated with the second user account.

In one or more embodiments, the series of acts 1000 includes determining the tree connection between the first user account and the second user account by comparing the genealogical tree associated with the first user account and a second genealogical tree associated with the second user account to identify the shared node and providing, on the client device associated with the first user account, the node suggestion and the shared node including an indication of the tree connection between the shared node and the node suggestion. In addition, in one or more embodiments, the series of acts 1000 includes receiving, from the client device associated with the first user account, a user interaction selecting the node suggestion and based on receiving the user interaction selecting the node suggestion, providing, on the client device associated with the client device, a visualization of the tree connection between the shared node, an intermediate node, and the node of the second user account indicated by the node suggestion.

Moreover, in one or more embodiments, the series of acts 1000 includes providing, on the client device associated with the first genetic profile, the option to display user accounts of the additional user accounts with genetic matches with the first genetic profile by providing the option with a node of the genealogical tree associated with the first user account and determining the tree connection between the first user account and the second user account by determining that the second user account has a tree connection to the node of the genealogical tree.

In addition, in one or more embodiments, the series of acts 1000 includes receiving, from the client device, a user interaction with the node suggestion to add the node suggestion to the genealogical tree and based on receiving the user interaction with the node suggestion, adding a node of the second user account to the genealogical tree associated with the first user account. Further, in one or more embodiments, the series of acts 1000 includes adding the node of the second user account to the genealogical tree by connecting the node of the second user account to a shared node.

Additionally, in one or more embodiments, the series of acts 1000 includes providing the node suggestion with an indication of the tree connection between the first user account and the second user account.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCS, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 11 illustrates a block diagram of an example computing device 1100 (e.g., the server(s) 102 and/or the client device 112) that may be configured to perform one or more of the processes described above. One will appreciate that one or more computing devices, such as the computing device 1100 may represent the computing devices described above (e.g., server(s) 102, client device 112, and computing server 800). In one or more embodiments, the computing device 1100 may be a mobile device (e.g., a mobile telephone, a smartphone, a PDA, a tablet, a laptop, a camera, a tracker, a watch, a wearable device, etc.). In some embodiments, the computing device 1100 may be a non-mobile device (e.g., a desktop computer or another type of client device). Further, the computing device 1100 may be a server device that includes cloud-based processing and storage capabilities.

As shown in FIG. 11, the computing device 1100 can include one or more processor(s) 1102, memory 1104, a storage device 1106, input/output interfaces 1108 (or “I/O interfaces 1108”), and a communication interface 1110, which may be communicatively coupled by way of a communication infrastructure (e.g., bus 1112). While the computing device 1100 is shown in FIG. 11, the components illustrated in FIG. 11 are not intended to be limiting. Additional or alternative components may be used in other embodiments. Furthermore, in certain embodiments, the computing device 1100 includes fewer components than those shown in FIG. 11. Components of the computing device 1100 shown in FIG. 11 will now be described in additional detail.

In particular embodiments, the processor(s) 1102 includes hardware for executing instructions, such as those making up a computer program. As an example, and not by way of limitation, to execute instructions, the processor(s) 1102 may retrieve (or fetch) the instructions from an internal register, an internal cache, memory 1104, or a storage device 1106 and decode and execute them.

The computing device 1100 includes memory 1104, which is coupled to the processor(s) 1102. The memory 1104 may be used for storing data, metadata, and programs for execution by the processor(s). The memory 1104 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1104 may be internal or distributed memory.

The computing device 1100 includes a storage device 1106 includes storage for storing data or instructions. As an example, and not by way of limitation, the storage device 1106 can include a non-transitory storage medium described above. The storage device 1106 may include a hard disk drive (HDD), flash memory, a Universal Serial Bus (USB) drive or a combination these or other storage devices.

As shown, the computing device 1100 includes one or more I/O interfaces 1108, which are provided to allow a user to provide input to (such as user strokes), receive output from, and otherwise transfer data to and from the computing device 1100. These I/O interfaces 1108 may include a mouse, keypad or a keyboard, a touch screen, camera, optical scanner, network interface, modem, other known I/O devices or a combination of such I/O interfaces 1108. The touch screen may be activated with a stylus or a finger.

The I/O interfaces 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1108 are configured to provide graphical data to a display for presentation to a user. The graphical data may be representative of one or more graphical user interfaces and/or any other graphical content as may serve a particular implementation.

The computing device 1100 can further include a communication interface 1110. The communication interface 1110 can include hardware, software, or both. The communication interface 1110 provides one or more interfaces for communication (such as, for example, packet-based communication) between the computing device and one or more other computing devices or one or more networks. As an example, and not by way of limitation, communication interface 1110 may include a network interface controller (NIC) or network adapter for communicating with an Ethernet or other wire-based network or a wireless NIC (WNIC) or wireless adapter for communicating with a wireless network, such as a WI-FI. The computing device 1100 can further include a bus 1112. The bus 1112 can include hardware, software, or both that connects components of computing device 1100 to each other.

FIG. 12 is a schematic diagram illustrating environment 1200 within which one or more implementations of the genetic-match-suggestion system 106 can be implemented. For example, the genetic-match-suggestion system 106 may be part of a genealogical-data system 104 (e.g., the genealogical-data system 104). The genealogical-data system 104 may generate, store, manage, receive, and send digital content (such as genealogical content items). For example, genealogical-data system 104 may send and receive digital content to and from client devices 1206 by way of network 1204. In particular, genealogical-data system 104 can store and manage genealogical databases for various user accounts, historical records, and genealogy trees. In some embodiments, the genealogical-data system 104 can manage the distribution and sharing of digital content between computing devices associated with user accounts. For instance, the genealogical-data system 104 can facilitate a user account sharing a genealogical content item with another user account of genealogical-data system 104.

In particular, the genealogical-data system 104 can manage synchronizing digital content across multiple client devices 1206 associated with one or more user accounts. For example, a user may edit a digitized historical document or a node within a genealogy tree using client device 1206. The genealogical-data system 104 can cause client device 1206 to send the edited genealogical content to the genealogical-data system 104, whereupon the genealogical-data system 104 synchronizes the genealogical content on one or more additional computing devices.

As shown, the client device 1206 may be a desktop computer, a laptop computer, a tablet computer, an augmented reality device, a virtual reality device, a personal digital assistant (PDA), an in- or out-of-car navigation system, a handheld device, a smart phone or other cellular or mobile phone, or a mobile gaming device, other mobile device, or other suitable computing devices. The client device 1206 may execute one or more client applications, such as a web browser (e.g., Microsoft Windows Internet Explorer, Mozilla Firefox, Apple Safari, Google Chrome, Opera, etc.) or a native or special-purpose client application (e.g., Ancestry: Family History & DNA for iPhone or iPad, Ancestry: Family History & DNA for Android, etc.), to access and view content over the network 1204.

The network 1204 may represent a network or collection of networks (such as the Internet, a corporate intranet, a virtual private network (VPN) , a local area network (LAN), a wireless local area network (WLAN), a cellular network, a wide area network (WAN), a metropolitan area network (MAN), or a combination of two or more such networks) over which client devices 1206 may access genealogical-data system 104.

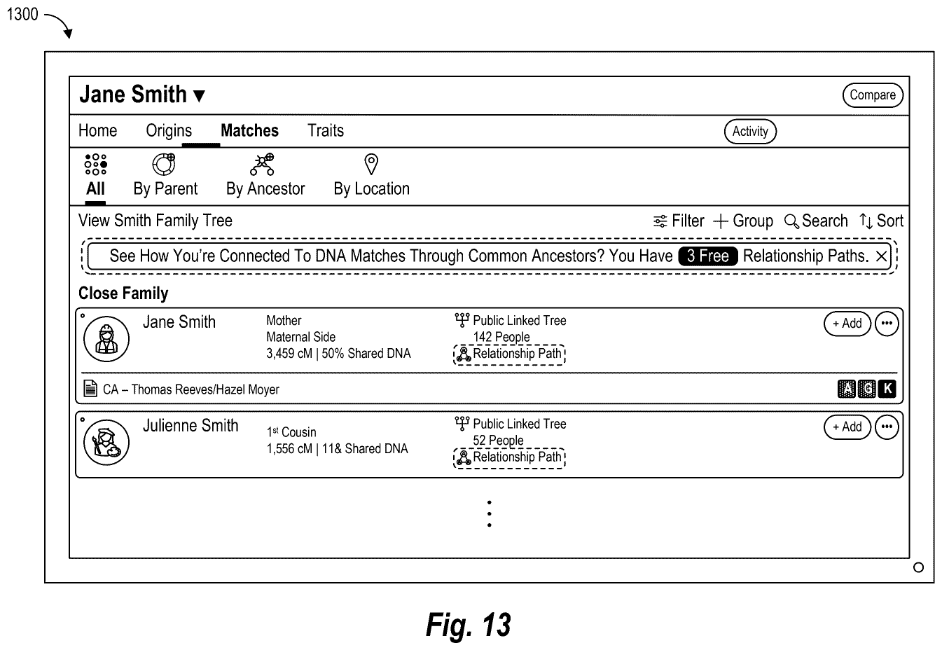

As seen in FIG. 13, a user interface 1300 may provide a user with options to view a relationship path between the user and one or more individuals or user accounts determined based on genetic similarity to be a relative. The relationship path for a particular relative may be determined using, e.g., a cluster database 914 as shown above in FIG. 9. That is, the cluster database 914, with its nodes and clusters, including at least one node in a cluster corresponding to the user and at least one node in a cluster corresponding to the relative, may be traversed by the genealogical-data system 104 to identify one or more most-efficient and/or highest-confidence paths between the clusters corresponding to the user and the relative, with the path defining a string of clusters and edges therebetween forming a connection between the clusters.

In embodiments, a confidence or predicted accuracy score associated with the one or more paths between the clusters is generated, the confidence score representing a likelihood that the path represents only accurate familial relationships. The likelihood may be determined based on one or more factors including, e.g., metadata associated with the clusters, a consistency between relationship predictions generated based on the genetic similarity (e.g. utilizing the number of shared segments, the cumulative cM shared, birth years, and/or other data as features for a trained machine learning model to predict the likely relationship between the individuals) and a genealogical definition of the relationship based on the relationship path, and/or any other suitable factor.

To elaborate, in embodiments, the DNA match may be predicted based on a particular number of shared segments, cumulative cM shared, and birth dates to be a user's third cousin. The genealogical-data system generates a relationship path between the user and the (potential) third cousin using, e.g., a cluster database to identify likely relationships in a universal genealogical tree and using suitable algorithms, models, or other approaches to optimize a path through the universal genealogical tree between the nodes. As a “third cousin” is a specific relationship involving specific ancestors (i.e. a great-great-grandparent, a great-grand-pibling, a first cousin twice removed, and a second cousin once removed), the genealogical-data system may be configured to compare the predicted relationship label with the relationship path to assess whether the relationship path traverses the requisite ancestors of both the user and the selected relative to be consistent with the predicted relationship label, with a confidence score generated based on whether and how precisely the path aligns with a predefined path through a family tree for that particular relationship label. Given that there may be a plurality of plausible paths through the universal genealogical tree between the clusters representing the user and the selected relative, the plurality of plausible paths may be ranked according to generated confidence scores.

The confidence scores may be generated using a heuristic-based approach similar to that described immediately above, and/or using a machine-learned modality that receives the DNA predicted relationship label and the generated relationship paths as features and is configured to output a classification and associated confidence regarding whether the relationship path is accurate based on the DNA match. Such a modality may advantageously be trained to account for the margins of error inherent in generating relationship predictions based on DNA alone and in predicting relationship paths from a cluster database. Other confidence-score-generation approaches are also contemplated.

In embodiments, a confidence score may include one or both of genetic and record-based validation or evidence for each node of a generated relationship path. For example, genetic evidence may include one or more factors including the user being related to others in their tree, e.g. based on sharing a certain threshold cM. In embodiments, a parental side through which the user shares a particular match may boost a confidence score of a node being placed on, e.g., the corresponding parental side of the user's tree. Record-based evidence may include records being attached to the person in the tree itself or to a corresponding cluster of a cluster database, but also records such as birth records that tie two nodes together.

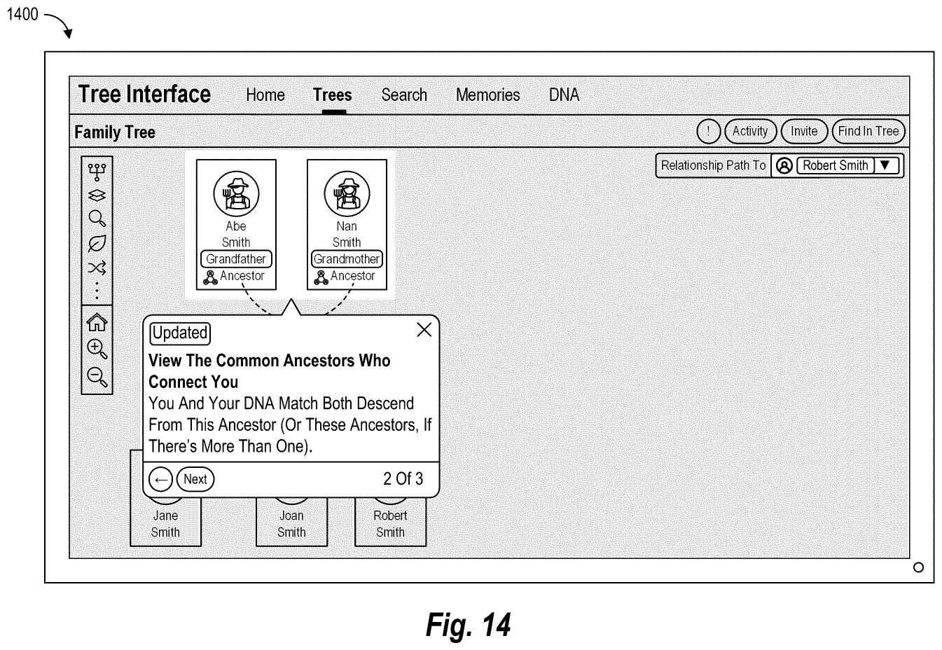

A user may select one an indicium associated with a relationship path for the one or more individuals shown in the user interface 1300, the indicium activating a subsequent user interface 1400 shown in FIG. 14. The user interface 1400 may include, in embodiments, a family-tree interface showing nodes representing the user, the selected relative, a most-recent common ancestor determined by the genealogical-data system 104 to be shared by the user and the selected relative. As seen, the family-tree interface shown in the user interface 1400 includes lines or other indicia representing the edges between the nodes representing the individuals with labels indicating the nature of the relationships represented by the edges, this allowing the user to more-intuitively understand and engage with the connection between the user and their selected relative. Thus, as seen in FIG. 14, the user can more intuitively appreciate that they are connected to the selected relative through their mother's brother.

Further, as described above, the family-tree interface of the user interface 1400 may include one or more intermediate individuals connecting the user and the selected relative to the most-recent common ancestor. As seen, one or more modals may be displayed, introducing the user to details regarding the predicted relationship path. For example, the modal may contextualize the predicted relationship, e.g. by populating the modal with predefined details regarding the relationship, such as third cousin. Additionally, or alternatively, the user interface 1400 may include an option for toggling the family-tree interface—and the depicted relationship path—between various relatives of the user interface 1300.

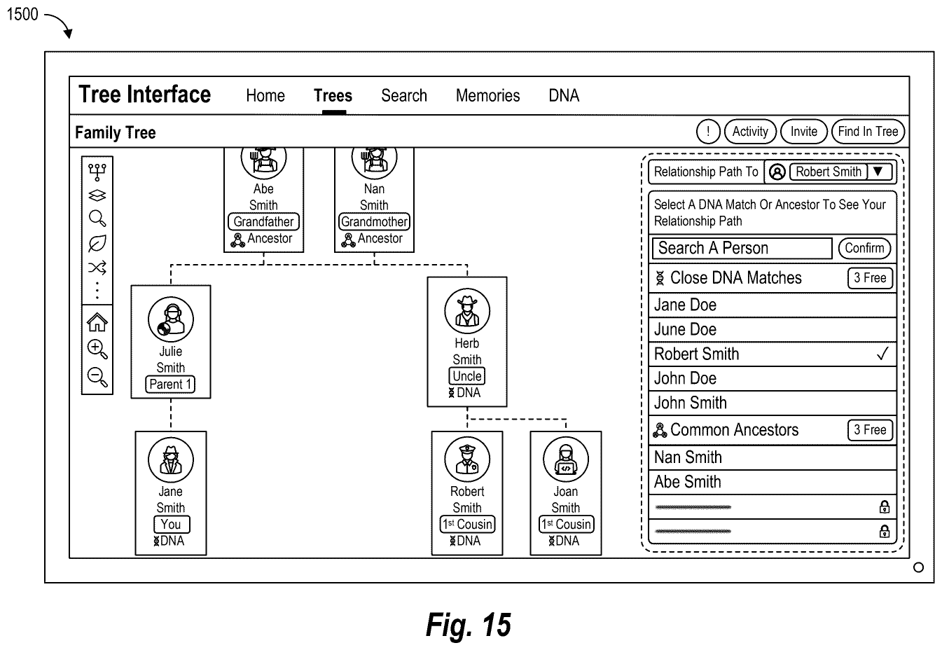

Turning now to FIG. 15, a user interface 1500 may show a family-tree interface including nodes for the user, the selected relative, the most-recent common ancestor, and any requisite intermediate nodes defining a particular relationship path between the user and the selected relative. As seen, the user interface 1500 may include a sidebar or other component displaying the available relatives between which the user may select or search. In embodiments, the sidebar displays available relatives in groups corresponding to shared common ancestors, though other possibilities are contemplated. The user may select between common ancestors of a plurality of common ancestors, thereby adjusting the displayed available relatives between which the user may select or search.

In embodiments, the relationship path displayed in the user interface 1500 may default to a highest-confidence or most-efficient path of a plurality of possible relationship paths between the user and the selected relative. In embodiments, the user interface 1500 may include an option for the user to toggle between or otherwise select other of the generated relationship paths.

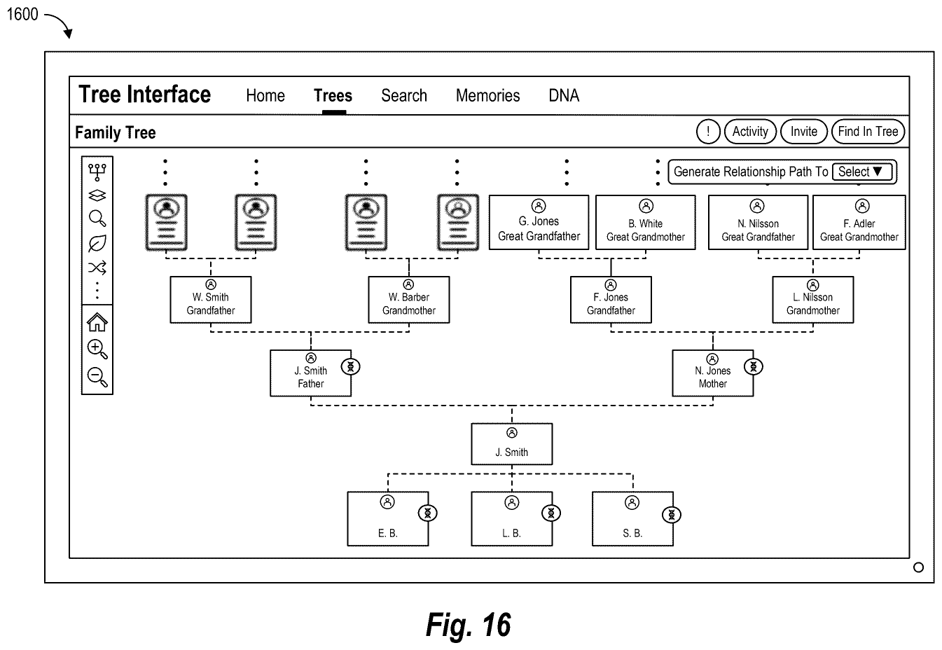

Turning now to FIG. 16, a user interface 1600 is shown, with an expanded family-tree interface that displays a plurality of relatives and ancestors of the user, with the node representing the user being arranged as the focal node. In the embodiment shown in FIG. 16, the user's ancestors are shown to a default vertical extent, such as great-grandparents. These terminal nodes may be associated with carets allowing the user to expand the family-tree interface further in a particular direction; for example, by selecting a caret associated with a particular one of the great-grandparents, the associated great-great-grandparent nodes may be shown in an expanded view of the family-tree interface. The nodes corresponding to ancestors may be labeled or otherwise be associated with indicia showing a number of DNA matches with the user sharing the pertinent ancestor as a most-recent common ancestor.

The user interface 1600 may transition to a subsequent user interface (not shown) upon a user selecting a node or associated indicium indicating a number of DNA matches of the user that share the ancestor node as a most-recent common ancestor. In embodiments, upon selecting the node or associated indicium indicating the number of DNA matches shared through a particular ancestor node, the family-tree interface may be expanded to populate the family-tree interface with the DNA matches of the user and determined intermediate nodes/individuals connecting the most-recent common ancestor to the DNA matches. Thus the selection of an ancestor node may expand the family-tree interface laterally and/or vertically; for example, upon selecting an indicium that a user has eight DNA matches through one of their maternal great-great-grandmothers, nodes corresponding to one or more, in embodiments each, of the eight DNA matches along with requisite intermediate nodes may be populated in the family-tree interface. By providing relationship paths on-demand in this manner, the user is better able to visualize and intuit the nature of the relationship between the user and the selected relative. That is, by starting from a particular shared ancestor, such as a maternal great-great-grandmother, and then being able to see a defined relationship path back down to the selected relative, the predicted relationship label “third cousin,” “second cousin once removed,” or otherwise can be better grasped, viewed, traversed, and appreciated in context. In contrast to a user being prompted to have interest in and select an option for viewing a relationship path between themselves and a DNA match of some unknown relationship such as “third cousin” in a bottom-up approach, the user interface 1600 has the advantage of utilizing a top-down approach, where a user may be presented with DNA relatives based on their sharing a particular ancestor. Indeed, a user may be more likely to engage with relationship paths with DNA relationships if they understand that they originate through their maternal great-great-grandmother, which is more intuitive than a floating third cousin.

Further, as seen, the user interface 1600 may include a toggle allowing a user to select between a plurality of DNA matches. The toggle may read “Generate relationship path to:” with the user allowed to select from a plurality of DNA matches, which may be sorted based on closest-predicted relationship, based on shared ancestor, or based on any other suitable organization.

The user interface 1600 further has the advantage of providing predicted tree interfaces for new users who may not have enough family-tree resources—e.g., a large-enough family tree and/or associated media files—to receive meaningful automated suggestions from or using the cluster database regarding a predicted relationship path between the user and selected DNA relative. This may be true in embodiments or situations where the cluster database relies for accuracy on a user's family tree having a critical mass of nodes, edges, and/or associated media files to have confidence in generated automated suggestions for further discovery for the user. Such new users may experience arrested development of their family tree, as they both lack the resources to continue building their tree and the genealogical-data service may lack the resources needed to meaningfully and accurately guide the user.

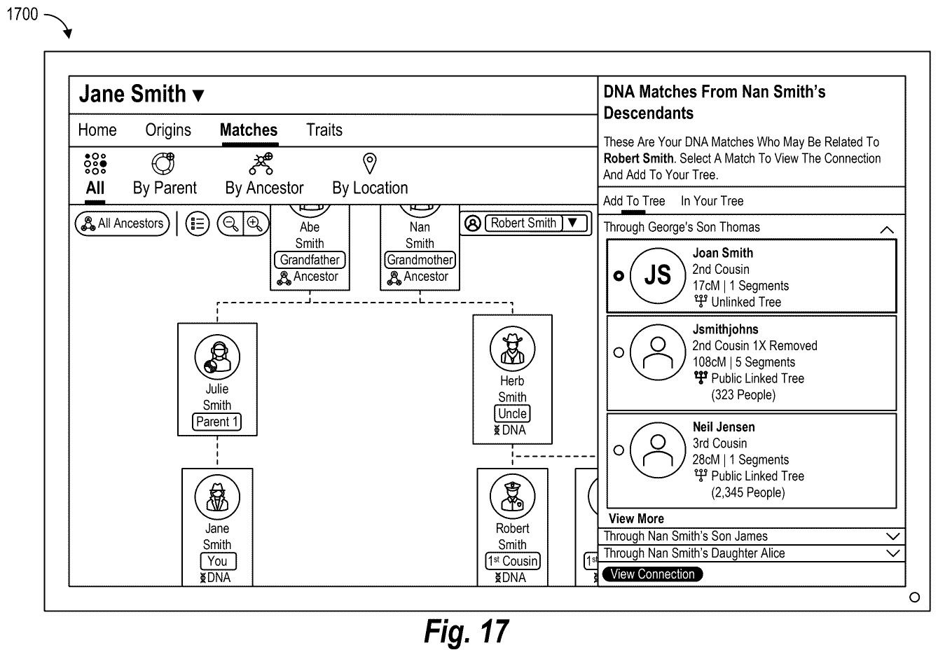

Turning now to FIG. 17, a user interface 1700 is shown. In an embodiment, upon selection by a user in the user interface 1600 of one of the ancestors with a plurality of DNA matches of the user descended therefrom, the user interface 1700 may drill down on the family-tree interface so as to focus the family-tree interface exclusively on the DNA matches that share that particular ancestor with the user. In this user interface 1700, a side panel may be generated to display a list of selectable relatives which may be added to the user's tree, along with, in embodiments, a discretization according to intermediate nodes. For instance, the selectable relatives may be grouped by children of the selected ancestor through whom the selectable relatives are connected to the user.

Each of the selectable relatives may be displayed with metadata facilitating informed choices by the user. For instance, the selectable relatives may include DNA-match metadata including predicted label (e.g. “second cousin”), number of shared segments, cumulative cM shared, and/or whether the selectable relative has an associate tree that the user can peruse to validate the relationship. The user interface 1700 may allow the user to select one of the displayed selectable relatives and to view the connection, e.g. by accessing a node or profile associated therewith.

In embodiments, users may be invited in one or more of the above-mentioned user interfaces and/or in a subsequent user interface to confirm, reject, adjust, or otherwise provide feedback on a suggested relationship and/or corresponding relationship path. For example, a generated relationship path may err in one of the intermediate nodes, indicating incorrectly that the DNA match is related to the most-recent common ancestor through a sibling of the actual ancestor. The user may adjust the node in the proposed relationship path by engaging with an edit indicium on a corresponding node and populating the correct node. In other embodiments, the user may correct a predicted relationship label and corresponding path based on a known relationship with the selected relative or based on correspondence between the user and the selected relative, in which correspondence the users are able to ascertain the precise nature of their relationship. In embodiments, the user feedback is as simple as a user checking a box if the relationship path appears to the user to be accurate and checking a different box if the relationship appears to be inaccurate. Other suitable modalities and examples of receiving user feedback via or in response to one or more of the generated relationship paths are also contemplated.

The feedback provided by users may be received and stored b the genealogical-data system in a suitable storage modality and utilized in, e.g., a reinforcement-learning human feedback loop to generate alternate possible relationship paths. Thus may be done in real-time or offline. Thus, for example, a user may provide feedback indicating that a proposed highest-confidence relationship path that connects the user with a particular selected relative is incorrect. The genealogical-data system may be configured to receive this feedback and, utilizing a suitable modality such as a reinforcement-learning model, to re-generate the relationship path, with the user again having an option to provide feedback on the regenerated relationship path. The reinforcement-learning model may learn and improve in real-time to provide improved relationship paths for a user, and more particularly over time in response to feedback from a plurality of users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

1. A computer-implemented method comprising:
generating, for a first user account of a genealogical-data system, a plurality of genetic-similarity predictions between a first genetic profile associated with the first user account and a plurality of genetic profiles associated with additional user accounts of the genealogical-data system;
determining, from the plurality of genetic-similarity predictions for the first user account, a genetic match indicating a second user account from the additional user accounts as matching the first user account;
based on determining the genetic match, determining a tree connection between the first user account and the second user account;
based on determining the tree connection between the first user account and the second user account, providing, on a client device associated with the first user account, a node suggestion to add a node for the second user account to a genealogical tree associated with the first user account; and
based on receiving an approval interaction with the node suggestion, adding the node corresponding to the second user account to the genealogical tree associated with the first user account.

2. The computer-implemented method of claim 1, wherein adding the node for the second user account to the genealogical tree associated with the first user account further comprises based on receiving the approval interaction with the node suggestion, modifying a cluster database storing clusters of genealogical records by linking a first cluster corresponding to the first user account with a second cluster corresponding to the second user account, wherein linking the first cluster with the second cluster associates the first user account with genealogical records corresponding to the second user account.

3. The computer-implemented method of claim 1, wherein determining the tree connection between the first user account and the second user account further comprises comparing the genealogical tree associated with the first user account and a second genealogical tree associated with the second user account to identify a shared node.

4. The computer-implemented method of claim 3, further comprising:
determining that there is a single-generation connection within the second genealogical tree between the second user account and the shared node; and
providing the node suggestion with an indication of the single-generation connection between the shared node and the second user account.

5. The computer-implemented method of claim 3, further comprising:
determining that the shared node is connected within the second genealogical tree to the second user account through an intermediate node; and
providing the node suggestion with an indication that the shared node is connected to the second user account through the intermediate node.

6. The computer-implemented method of claim 5, further comprising:
receiving a user interaction to add the intermediate node to the genealogical tree associated with the first user account;
based on receiving the user interaction to add the intermediate node to the genealogical tree, adding the intermediate node to the genealogical tree; and
based on receiving the approval interaction to add the node for the second user account to the genealogical tree and further based on adding the intermediate node to the genealogical tree, adding the node for the second user account to the genealogical tree.

7. The computer-implemented method of claim 1, further comprising:
based on receiving the approval interaction with the node suggestion to add the node for the second user account to the genealogical tree, providing a genealogical record on the client device;
receiving, from the client device, a user interaction confirming that the genealogical record is associated with a shared node; and
adding the node for the second user account to the genealogical tree based on receiving the user interaction confirming that the genealogical record is associated with the shared node.

8. The computer-implemented method of claim 1, wherein providing the node suggestion further comprises:
determining that the tree connection between the first user account and the second user account comprises an intermediate node that corresponds to a user account of a living person;
based on determining that the tree connection between the first user account and the second user account comprises an intermediate node that corresponds to the user account of the living person, providing the node suggestion comprising a suggestion to add the intermediate node to the genealogical tree as a living person node without including personal information of the living person; and
providing, on the client device associated with the first user account, an option to communicate with the second user account.

9. The computer-implemented method of claim 1, wherein providing the node suggestion further comprises providing the node suggestion with an option to communicate with the second user account.

10. A non-transitory computer-readable medium storing instructions that, when executed by at least one processor, cause a computer system to:
generate a plurality of genetic-similarity predictions between a first genetic profile associated with a first user account of a genealogical-data system and a plurality of genetic profiles associated with additional user accounts of the genealogical-data system;
determine, from the plurality of genetic-similarity predictions for the first user account, a genetic match indicating a second user account from the additional user accounts as matching the first user account based on a genetic-similarity prediction between the first genetic profile and a second genetic profile associated with a second user account;
based on determining the genetic match, determine a tree connection between the first user account and the second user account;
based on determining the tree connection between the first user account and the second user account, provide, on a client device associated with the first user account, a node suggestion to add a node for to the second user account to a genealogical tree associated with the first user account and an indication of a shared node between the first user account and the second user account; and
based on receiving an approval interaction with the node suggestion, add the node for the second user account to the genealogical tree associated with the first user account.

11. The non-transitory computer-readable medium of claim 10, further comprising instructions that, when executed by the at least one processor, cause the computer system to:
receive, from the client device, a user interaction with an option to display user accounts having genetic matches to the shared node of the genealogical tree of the first user account; and
provide the node suggestion and the indication of the shared node in response to receiving the user interaction to display suggested user accounts.

12. The non-transitory computer-readable medium of claim 10, further comprising instructions that, when executed by the at least one processor, cause the computer system to based on receiving the approval interaction with the node suggestion, modify a cluster database storing clusters of genealogical records by linking a first cluster corresponding to the first user account with a second cluster corresponding to the second user account, wherein linking the first cluster with the second cluster associates the first user account with genealogical records corresponding to the second user account.

13. The non-transitory computer-readable medium of claim 12, further comprising instructions that, when executed by the at least one processor, cause the computer system to add the node for the second user account to the genealogical tree by adding a username, a gender, and a person status associated with the second user account.

14. The non-transitory computer-readable medium of claim 10, further comprising instructions that, when executed by the at least one processor, cause the computer system to determine the tree connection between the first user account and the second user account by:
comparing the genealogical tree associated with the first user account and a second genealogical tree associated with the second user account to identify the shared node; and
providing, on the client device associated with the first user account, the node suggestion and the shared node including an indication of the tree connection between the shared node and the node suggestion.

15. The non-transitory computer-readable medium of claim 14, further comprising instructions that, when executed by the at least one processor, cause the computer system to:
receive, from the client device associated with the first user account, a user interaction selecting the node suggestion; and
based on receiving a user interaction with the node suggestion, providing, on the client device associated with the client device, a visualization of the tree connection between the shared node, an intermediate node, and the node suggestion.

16. A system comprising:
at least one processor; and
at least one non-transitory computer-readable storage medium storing instructions that, when executed by the at least one processor, cause the system to:
generate a plurality of genetic-similarity predictions between a first genetic profile associated with a first user account of a genealogical-data system and a plurality of genetic profiles associated with additional user accounts of the genealogical-data system;
based on receiving a user interaction with an option to display genetic matches with the first genetic profile, determine, from the plurality of genetic-similarity predictions for the first user account, a genetic match indicating a second user account from the additional user accounts as matching the first user account based on a genetic-similarity prediction between the first genetic profile and a second genetic profile associated with a second user account;
based on determining the genetic match, determine a tree connection between the first user account and the second user account;
based on determining the tree connection between the first user account and the second user account, provide, on a client device associated with the first user account, a node suggestion to add a node for the second user account to a genealogical tree associated with the first user account; and
based on receiving an approval interaction with the node suggestion, add the node for to the second user account to the genealogical tree associated with the first user account.

17. The system of claim 16, further comprising instructions that, when executed by the at least one processor, cause the system to:
provide, on the client device associated with the first genetic profile, the option to display user accounts of the additional user accounts with genetic matches with the first genetic profile by providing the option with a node of the genealogical tree associated with the first user account; and
determine the tree connection between the first user account and the second user account by determining that the second user account has a tree connection to the node of the genealogical tree.

18. The system of claim 16, further comprising instructions that, when executed by the at least one processor, cause the system to add the node for the second user account to the genealogical tree associated with the first user account by, based on receiving the approval interaction with the node suggestion, modifying a cluster database storing clusters of genealogical records by linking a first cluster corresponding to the first user account with a second cluster corresponding to the second user account, wherein linking the first cluster with the second cluster associates the first user account with genealogical records corresponding to the second user account.

19. The system of claim 16, further comprising instructions that, when executed by the at least one processor, cause the system to add the node for the second user account to the genealogical tree by connecting the node of the second user account to a shared node.

20. The system of claim 16, wherein a confidence score is generated for a relationship path corresponding to the tree connection between the first user account and the second user account, wherein determining the confidence score includes:
predicting, based on DNA data, a relationship label for a relationship between the first user account and the second user account;
comparing the relationship label to a relationship path corresponding to the tree connection; and
determining the confidence score based on comparing the relationship label to the relationship path corresponding to the tree connection.
